(19) United States
(12) Patent Application Publication
(10) Pub. No.: US 2009/0318534 A1
(43) Pub. Date: Dec. 24, 2009

(54) METHODS AND COMPOSITIONS FOR THE TREATMENT OF SKIN DISEASES AND DISORDERS

(75) Inventors: Richard L. Gallo, San Diego, CA (US); Jurgen Schauber, La Jolla, CA (US); Kenshi Yamasaki, San Diego, CA (US)

Correspondence Address: Joseph R. Baker, APC;Gavrilovich, Dodd & Lindsey LLP, 4660 La Jolla Village Drive, Suite 750, San Diego, CA, 92122, US

(73) Assignee: THE REGENTS OF THE UNIVERSITY OF CALIFORNIA, Oakland, CA (US)

(21) Appl. No.: 12/443,312

(22) PCT Filed: Sep. 26, 2007
(86) PCT No.: PCT/US07/21029

§ 371 Date: Mar. 27, 2009

Related U.S. Application Data
(60) Provisional application No. 60/847,877, filed on Sep. 27, 2006.

(51) Int. Cl. A61K 48/00; A61P 17/10; A61P 17/00; G01N 33/53; C12Q 1/68

(52) U.S. Cl. 514/44 A; 514/44 R; 435/29; 435/23; 435/6

(57) ABSTRACT

The disclosure demonstrates the role of cathelicidin, serine protease and/or vitamin D3 in rosacea pathology.

### CROSS REFERENCE TO RELATED APPLICATIONS

This application claims priority under 35 U.S.C. §119 from Provisional Application Ser. No. 60/847,877, filed Sep. 27, 2006, the disclosure of which is incorporated herein by reference.

### TECHNICAL FIELD

The disclosure relates to methods and compositions for treating skin diseases and disorder and more specifically to methods and compositions for treating rosacea and acne.

### BACKGROUND

Rosacea is a chronic skin condition characterized by recurrent episodes of flushing, erythema, vasodilation, telangiectasia, edema, papules, pustules, hyperplasia, fibroplasia, itching, burning, pain, and skin tightness. Symptoms of rosacea are exacerbated by sun exposure, hot weather, immersion in hot water, high humidity, sweating, exercise, emotional stress, and spicy food. The skin condition usually begins between the ages of 30 to 50 and occurs more frequently in women than men.

The etiology of rosacea is not well understood, but it has been presumed to be caused by an as yet unidentified infectious agent. Unfortunately, antibiotic administration yields only marginal improvement.

As a member of the resident human microflora, the Gram-positive anaerobic coryneform bacterium Propionibacterium acnes is found predominantly in the sebaceous gland of the skin. It can, however, also be isolated from the conjunctiva, the external ear canal, the mouth, the upper respiratory tract and, in some individuals, the intestine. P. acnes has an estimated skin density of 10² to 10⁵⁻⁶ cm⁻². P. acnes is a well-recognized opportunistic pathogen, especially in relation to medical implants such as central nervous system shunts, silicone implants and prosthetic hip joints. It is also responsible for ocular and periocular infections and endophthalmitis and has been implicated in periodontal and dental infections. Dental probing and treatment has lead to the dissemination of P. acnes in the bloodstream, which is a recognized cause of endocarditis in relation to damaged or prosthetic heart valves. P. acnes also plays a role in inflammatory acne, since antimicrobial therapy directed against P. acnes results in improvement, while the development of antibiotic resistance in P. acnes is associated with relapse. The common form of acne, known as acne vulgaris, affects up to 80% of the population at some time in their lives, making it the most common skin infection. There is also a strong association between severe forms of acne and joint pain, inflammation of the bone (osteitis) and arthritis. In patients suffering from this condition, known as SAPHO (synovitis, acne, pustulosis, hyperostosis and osteitis) syndrome, isolates of P. acnes have been recovered from bone biopsy samples, as well as synovial fluid and tissue.

### SUMMARY

The disclosure provides a method of treating skin diseases and disorders such as inflammatory diseases and disorders, rosacea and acne comprising administering a cathelicidin inhibitor. In one aspect, the cathelicidin inhibitor comprises an antisense or ribozyme molecule that inhibits the expression of a cathelicidin polypeptide. In another aspect, the cathelicidin inhibitor comprises a vitamin D3 antagonist. In yet another aspect, the cathelicidin inhibitor comprises a serine protease inhibitor. The administering can be by topical application.

The disclosure also provides a composition comprising a cathelicidin antisense or ribozyme molecule, a vitamin D3 antagonist and/or a protease inhibitor.

The disclosure also provides a method for determining whether a subject has or is at risk of having rosacea comprising determining the level of a cathelicidin polypeptide and/or serine protease in a sample from the subject, wherein an elevate level of cathelicidin is indicative of rosacea or risk of having rosacea. In one aspect, the cathelicidin polypeptide is LL-37 and/or FA-29.

In another aspect, the serine protease is kallikrein SCTE. The disclosure also provides a method for determining whether a subject has or is at risk of having rosacea comprising determining polypeptide mass in a sample from the subject, wherein an elevate level of mass is indicative of rosacea or risk of having rosacea.

A kit comprising a reagent useful for identifying the level of cathelicidin and/or serine protease in a skin sample. The reagent may be an anti-cathelicidin antibody (e.g., an anti-LL-37 antibody, or an anti-FA29 antibody). The reagent may be a nucleic acid probe capable of hybridizing to a cathelicidin-encoding nucleic acid.

The details of one or more embodiments of the disclosure are set forth in the accompanying drawings and the description below. Other features, objects, and advantages of the disclosure will be apparent from the description and drawings, and from the claims.

### BRIEF DESCRIPTION OF THE DRAWINGS

FIG. 1A-G shows that cathelicidin is abundant in rosacea. hCAP18/LL-37 expression in lesional skin of rosacea patients were examined with immunohistochemistry. a, b, d, e: rosacea lesional skin, c, f: normal skin, a-c: anti-LL-37 antibody, d-f: preimmune serum. Original magnification: a, d: 10×, and b, c, e, f: 40×. g: hCAP18/LL-37 in skin was measured by quantitative immunodot blot of tape stripped samples. Average of each group is indicated with broken lines (n=11). h-k: Localization of cathelicidin mRNA in lesional skin of rosacea individuals was visualized by in situ hybridization with a probe to LL-37. Brown color indicates positive signal; blue color indicates methylene blue staining of nuclei. Left, antisense probe; right, sense probe. Scale bars, 500 μm.

FIG. 2 shows altered cathelicidin peptides expression in rosacea skin. Mass spectrum of cathelicidin peptides in lesional skin of individual rosacea patients (upper three columns) and in 3 normal patients (lower three columns) examined by SELDI-TOF-MS system. Arrows indicate unique peaks in rosacea skin.

FIG. 3A-K shows increased stratum corneum tryptic enzyme (SCTE) expression and protease activity in rosacea epidermis. a-f: Expression of cathelicidin and SCTE in skin visualized by immunofluorescence. a-c: rosacea, d-f normal skin. Green indicates cathelicidin and red indicates SCTE. Original magnification: 10×. g-j: Protease activity in human skin examined by in situ zymography with FITC-conjugated casein substrate (g, i). More intense green signal corresponds to increase proteolysis. Nuclei are stained blue with DAPI (h, j). Original magnification: 10×. k: Total proteolytic activity of rosacea skin extracts measured in solution by fluorescence emission of FITC-conjugated casein substrate. Samples treated with various protease inhibitors show that addition of serine protease inhibitors aprotinin or AEBSF are effective in eliminating activity.

FIG. 4A-L shows cathelicidin peptides augment cytokine induction in human keratinocytes and skin inflammation. a: Human epidermal keratinocytes were stimulated by cathelicidin peptides at indicated concentrations for 6 h and IL-8 secretion in cultured media were measured by ELISA. The average and S.E.M. of three experiments each done in triplicate are shown. b-e: After injection of cathelicidin peptides (320 μM in 40 μl) twice a day for 2 d, mouse skin inflammation was evaluated. One representative image of skin surface and histology from three independents experiments are shown. b, d: LL-37 injected skin, c, e: KR-20 injected skin. f-h: Skin irritation was caused by epicutaneous application of 2% DNFB to the back of mCRAMP knockout (Cnlp^{-/-}) mice (f) and WT littermates (Cnlp +/+) mice. Five days after application, mouse skin was biopsied and inflammation evaluated histologically and leukocytes counted. The mean and S.D. of leukocytes per high power field (HPF) from three randomly selected regions are plotted on the graph (h). (i) Cathelicidin processing in Spink5^{-/-} mice analyzed by SELDI-TOF-MS. Skin from Spink5^{-/-} mice had processed cathelicidin peptides of <7 kDa (top), whereas the main cathelicidin in wild-type (Spink5^{+/+}) mice was a non-processed form of >8 kDa (bottom). Arrowhead indicates GLL-34, a representative mouse cathelicidin peptide. (j) SCTE or boiled SCTE was injected subcutaneously, and histology (left) and cathelicidin processing (right) were examined. SCTE-treated skin showed inflammation and processed cathelicidin peptide (m/z 4,244, sequence FKKISRLAGLLRKGGEKIGEKLKKIGQKIKNFFQKLV (SEQ ID NO:3)). Scale bars, 500 μm. (k) SCTE was injected subcutaneously into Camp^{-/-} and wild-type mice. Skins were processed by hematoxylin-eosin staining (k), and the mean±s.d. (n=4) of infiltrated cells per HPF was plotted (h). Camp^{-/-} mice showed significantly less cell infiltration (P<0.05).

FIG. 5 shows cathelicidin peptide expression in rosacea. Mass sizes of cathelicidin peptides in lesion skin of rosacea were examined by SELDI-TOF-MS system. Deduced peptide sequences (SEQ ID NOs:4-14), abbreviation of peptides, and mass sizes of each peak are shown in the table.

FIG. 6A-B shows increased SCTE expression and protease activity in rosacea. a, b: Expression of cathelicidin and SCTE in skin are visualized by immunofluorescence. a: rosacea skin from 5 individuals (R1-R5), b: normal skin from 5 individuals (N1-N5). Nomarski differential interference contrast images obtained under same field are shown in a right column. Original magnification: 10×.

FIG. 7A-D shows cathelicidin peptides induce neutrophil infiltration and increased microvessels. a, b: LL-37- and PBS-injected mouse skin were stained with anti-mouse Gr-1 (a) and anti-mouse CD31 (PECAM) (b) to exam neutrophil infiltration and generation of microvessels, respectively. Nuclei are stained with DAPI. c: After injection of cathelicidin peptides FA-29 (320 μM in 40 μl) or PBS (vehicle, 40 μl) twice a day for 2 d, mouse skin inflammation was monitored and biopsied. The biopsies were processed for hematoxylin-eosin staining and histopathological analysis. One representative image of skin surface and histology of each skin from at least three independents experiments are shown. Left column: FA-29 injected skin, right column: PBS injected skin. d: LL-37 (320-0.32 μM, 40 μl) was injected twice a day for 2 d and the skin inflammation was monitored. Representative images of skin area injected with indicated doses of LL-37 are shown.

### DETAILED DESCRIPTION

Unless defined otherwise, all technical and scientific terms used herein have the same meaning as is commonly understood by one of skill in the art to which this disclosure belongs. All publications and patents referred to herein are incorporated by reference.

As used herein and in the appended claims, the singular forms “a,” “and,” and “the” include plural referents unless the context clearly dictates otherwise. Thus, for example, reference to “a cell” includes a plurality of such cells and reference to “the peptide” includes reference to one or more peptides known to those skilled in the art, and so forth.

The publications discussed above and throughout the text are provided solely for their disclosure prior to the filing date of the present application. Nothing herein is to be construed as an admission that the inventors are not entitled to antedate such disclosure by virtue of prior disclosure.

As used herein, the term “skin” refers to the outer protective covering of the body of a mammal (e.g., a human), consisting of the corium and the epidermis, and is understood to include sweat and sebaceous glands, as well as hair follicle structures. Throughout the disclosure, the adjective “cutaneous” can be used, and should be understood to refer generally to attributes of the skin, as appropriate to the context in which they are used.

The methods and compositions of the disclosure find use in the treatment of cutaneous inflammatory diseases and disorders. For example, serine proteases and known to play a role in inflammation. Cells that produce serine proteases, e.g., monocytes, as well as monocyte-derived macrophages and dendritic (Langerhans) cells, have important roles in many autoimmune diseases, such as psoriasis, atopic dermatitis, pemphigus vulgaris, and lupus dermatitis. Various forms of acne (e.g., acnes vulgaris and acnes rosacea) have inflammatory components to their etiology.

Rosacea is a common facial dermatitis that currently affects an estimated 13 million Americans. It is a chronic and progressive cutaneous vascular disorder, primarily involving the malar and nasal areas of the face. Rosacea is characterized by flushing, erythema, papules, pustules, telanglectasia, facial edema, ocular lesions, and, in its most advanced and severe form, hyperplasia of tissue and sebaceous glands leading to rhinophyma. Rhinophyma, a florid overgrowth of the tip of the nose with hypervascularity and modularity, is an unusual progression of rosacea of unknown cause. Ocular lesions are common, including mild conjunctivitis, burning, and grittiness. Blepharitis, the most common ocular manifestation, is a nonulcerative condition of the lid margins.

Rosacea most commonly occurs between the ages of 30 to 60, and may be seen in women experiencing hormonal changes associated with menopause. Women are more frequently affected than men; the most severe cases, however, are seen in men. Fair complexioned individuals of Northern European descent are most likely to be at risk for rosacea; most appear to be pre-disposed to flushing and blushing. Although papules and pustules are associated with rosacea, and hence its misnomer as “acne rosacea”, the occurrence of P. acnes is generally not associated with the condition.

The cause of rosacea is poorly understood, numerous theories have been offered. Hypotheses have included gastrointestinal, psychological, infectious, climatic, and immunological causes. A commonly proposed etiologic theory is based on the presence of Demodex folliculorum mites in patients with rosacea; the organism feeds on sebum, and in some cases treatment of demodex infestation have provided improvement in the rosacea; however, in a review of biopsies, demodex folliculorum was noted in only 19% of the specimens. A bacterial cause for the disease has been hypothesized, but no consistent findings of one bacteria have been demonstrated. Climate, specifically exposure to extremes of sun and cold, may have an effect on the course of the disease, but the role of climate is not clear. An autoimmune process has been suggested, and tissue fixed immunoglobulins have been reported in patients with chronic inflammation of rosacea, but no other evidence has been found. Other experimental evidence has suggested this disease may represent a type of hypersensitivity reaction. No single hypothesis appears to adequately explain both the vascular changes and the inflammatory reaction seen in rosacea, leaving the pathogenesis unclear.

Histopathologic findings in rosacea dermatitis include vascular dilatation of the small vessels with perivascular infiltration of histiocytes, lymphocytes, and plasma cells. Dermal changes include loss of integrity of the superficial dermal connective tissue with edema, disruption of collagen fibers, and frequently severe elastosis. Follicular localization is infrequent and, when seen, is usually manifest clinically as pustules. However, there is no primary follicular abnormality. Immunoglobulin and compliment deposition at the dermal-epidermal junction have been reported in conjunctival and skin biopsies from rosacea patients. Ocular pathologic findings include conjunctival and corneal infiltration with chronic inflammatory cells, including lymphocytes, epithelioid cells, plasma cells, and giant cells.

The methods and compositions of the disclosure also have use in the treatment of other skin diseases and disorders including, for example, acne (including acne rosacea and acne vulgaris). Two distinct phenotypes of P. acnes, types I and II, have been identified based on serological agglutination tests and cell-wall sugar analysis. Recently, recA-based sequence analysis has revealed that P. acnes types I and II represent phylogenetically distinct groups (McDowell et al., 2005).

P. acnes produces a co-haemolytic reaction with both sheep and human erythrocytes (Choudhury, 1978) similar to the Christie-Atkins-Munch-Petersen (CAMP) reaction first demonstrated in 1944 (Christie et al., 1944). The CAMP reaction describes the synergistic haemolysis of sheep erythrocytes by the CAMP factor from Streptococcus agalactiae and the -toxin (sphingomyelinase C) from Staphylococcus aureus, with the CAMP factor demonstrating non-enzymatic affinity for ceramide (Bernheimer et al., 1979). Examination of sphingomyelinase-treated sheep erythrocytes has revealed the formation of discrete membrane pores by recombinant Streptococcus agalactiae CAMP factor (Lang & Palmer, 2003). In addition to the extensive study of the CAMP factor of Streptococcus agalactiae (Bernheimer et al., 1979; Brown et al., 1974; Jurgens et al., 1985, 1987; Ruhlmann et al., 1988; Skalka et al., 1980), a number of other Gram-positive and Gram-negative bacteria are known to produce a positive CAMP reaction, including Pasteurella haemolytica (Fraser, 1962), Aeromonas species (Figura and Guglielmetti, 1987), some Vibrio species (Kohler, 1988) and group G streptococci (Soedermanto and Lammler, 1996). Some of these species can also use phospholipase C (-toxin) from Clostridium perfringens or phospholipase D from Corynebacterium pseudotuberculosis as a co-factor for haemolysis in addition to the Staphylococcus aureus-toxin (Frey et al., 1989). The CAMP factor genes of Actinobacillus pleuropneumoniae and Streptococcus uberis have also been identified, cloned and expressed in Escherichia coli (Frey et al., 1989; Jiang et al., 1996).

The disclosure demonstrates that individuals with rosacea express abnormally high levels of cathelicidin in their facial skin and that the proteolytically processed forms of cathelicidin peptides found in rosacea are increased and/or different from those present in normal individuals. These cathelicidin peptides are a result of a post-translational processing abnormality associated with an increase in stratum corneum tryptic enzyme (SCTE) in the epidermis. Experiments in which cathelicidin peptides were injected cutaneously into a subject in combination with an increased protease activity, by targeted deletion of the serine protease inhibitor gene Spink5 increased inflammation in mouse skin. The role of cathelicidin in enabling SCTE-mediated inflammation was verified in mice with a targeted deletion of Camp, the gene encoding cathelicidin. This data confirms the role of cathelicidin in skin inflammatory responses and provides an explanation for the pathogenesis of rosacea.

The disclosure is based, in part, upon abnormal proteolytic processing as an etiologic explanation for rosacea and provides a therapeutic approach to this disorder. This is the first time that rosacea has been linked to altered levels of expression of cathelicidin, or its proteolytic enzymes, in skin. Skin of subjects with rosacea express more cathelicidin than normal facial skin. Additionally, levels of the cathelicidin precursor protein hCAP18, cathelicidin peptides LL-37 and FA-29, and the serine protease kallikrein (SCTE) are significantly higher in rosacea skin than in normal skin. Rosacea skin contains peptides of unique mass that are absent from normal skin, as determined by mass spectrometry (SELDI-TOF-MS).

Cathelicidin proteins are composed of two distinct domains: an N-terminal “cathelin-like” or “prosequence” domain and the C-terminal domain of the mature anti-microbial peptide (AMP). The C-terminal domain of cathelicidins was among the earliest mammalian AMPs to show potent, rapid, and broad-spectrum killing activity. The term “cathelin-like” derives from the similarity of the N-terminal sequence with that of cathelin, a 12 kDa protein isolated from porcine neutrophils that shares similarity with the cystatin superfamily of cysteine protease inhibitors.

Cathelicidins are expressed in neutrophils and myeloid bone marrow cells and most epithelial sources, and were the first AMPs discovered in mammalian skin due to their presence in wound fluid. In the neutrophil, cathelicidins are synthesized as full-length precursor and targeted to the secondary granules where they are stored. Upon stimulation, the full-length cathelicidin protein is proteolytically processed to unleash the microbialcidal activity of the C-terminal peptide from the cathelin-like domain. Human cathelicidin antimicrobial peptide (CAMP) gene is a direct target of the vitamin D receptor and is strongly up-regulated in myeloid cells by 1,25-dihydroxyvitamin D3 (see, e.g., FASEB Journal, 19:1067-1077, 2005).

Vitamin D3 leads to increased expression of Toll-like receptor 2 (TLR2) and CD14, which in turn induce antimicrobial peptides. Thus, Vitamin D3 both induces cathelicidin and enables TLR2 responsiveness to further increase expression of cathelicidin. For example, normal keratinocytes stimulated with Vitamin D3 show induced the expression of cathelicidin in normal human keratinocytes as well as the keratinocytic cell line HaCat. A vitamin D3 response element in the cathelicidin promoter was necessary for cathelicidin production. In particular, 1,25 OH D3 induces the expression of LL-37.

Vitamin D3 is produced from dietary or endogenous precursors under the influence of UVB light. Activation of vitamin D3 to 1,25 OH D3 requires two major hydroxylation steps, the first by 25-hydroxylase (CYP27A1) and then by 1α-hydroxylase (CYP27B1). These enzymes are mainly located in the human liver and kidney, respectively. However, some 1,25 OH D3 targeted organs such as the epidermis also posses the enzymes to produce 1,25 OH D3. Upon binding to the vitamin D receptor (VDR), 1,25 OH D3 activates target genes through vitamin D responsive elements in the gene promoter. Simultaneously, 1,25 OH D3 induces the vitamin D3 catabolic enzyme CYP24A1 (24-hydroxylase) thereby initializing its own degradation. Control of 1,25D3 producing and catabolizing enzymes therefore determines the level of bioactive hormone.

1,25 OH D3 hydroxylase, and specific receptors in several tissues, capable of converting non-active vitamin D3 to active 1,25 OH D3 are found in such tissues as bone, keratinocytes, placenta, and immune cells. Accordingly, inhibiting the activity of such enzymes may prove useful for treating inflammatory diseases and disorders of the epithelium (e.g., rosacea, acnes and the like). Furthermore, increased catabolic activity that degrades active vitamin D3 can be used to treat such diseases and disorders (e.g., rosacea). For example, stimulating the vitamin D3 catabolic enzyme CYP24A1 can reduce the amount of vitamin D3 present in the skin and thereby reduce the stimulatory effect vitamin D3 has on cathelicidin production.

The compositions and methods of the disclosure utilize Vitamin D3 antagonists alone, protease inhibitors alone, vitamin D3 catabolic enzymes alone, cathelicidin inhibitors or various combinations thereof to treat inflammation, rosacea and acnes. The C-terminal 37 amino acids of human cathelicidin (LL-37) has been characterized. LL-37 was originally referred to as FALL39, named for the first 4 N-terminal amino acids of this domain and the total number of residues (i.e., 39). LL-37 is a peptide predicted to contain an amphipathic alpha helix and lacks cysteine, making it different from all other previously isolated human peptide antibiotics of the defensin family, each of which contain 3 disulfide bridges. Full length human cathelicidin (sometimes referred to as full length LL-37) comprises the cathelin-like precursor protein and the C-terminal LL-37 peptide, thus comprising 170 amino acids (SEQ ID NO:2).

The polypeptide comprising SEQ ID NO:2 has a number of distinct domains. For example, a signal domain comprising a sequence as set forth from about 1 to about 29-31 of SEQ ID NO:2 is present. The signal domain is typically cleaved following amino acid number 30 of SEQ ID NO:2, however, one of skill in the art will recognize that depending upon the enzyme used, the expression system used and/or the conditions under which proteolytic cleavage of the polypeptide takes place, the cleavage site may vary from 1 to 3 amino acid in either direction of amino acid number 30 of SEQ ID NO:2. Another domain comprises the N-terminal domain, referred to as the cathelin-like domain. The cathelin-like domain comprises from about amino acid 29 (e.g., 29-31) to about amino acid 128 (e.g., 128-131) of SEQ ID NO:2. Yet another domain of SEQ ID NO:2 comprises the C-terminal domain referred to as LL-37. The LL-37 domain comprises from about amino acid 128 (e.g., 128-134) to amino acid 170 of SEQ ID NO:2. The full length LL-37 polypeptide is set forth in SEQ ID NO:2

The human cDNA sequence for full length LL-32 is set forth in SEQ ID NO:1. The coding sequence of an active fragment of LL-37 can be identified with reference to the cDNA sequence provided in SEQ ID NO:1 without difficulty. Accordingly the corresponding coding sequences of the fragments identified herein are also provided by the disclosure. The development of antisense and ribozyme molecules useful in the methods and compositions of the invention can be readily identified based upon the sequence listing provided herein as well as reference to variants and homologs known in the art.

The disclosure provides methods and compositions useful for the treatment of inflammatory diseases and disorders of the skin including, but not limited to, rosacea and acnes. For example, a drug that targets and inhibits cathelicidin proteolysis or reduction in cathelicidin production or activity provides an effective treatment of rosacea. One can treat rosacea by inhibiting cathelicidin expression through topical inhibition of Vitamin D or the Vitamin D receptor to reduce up regulation of cathelicidin, or inhibit the kallikrein stratum corneum tryptic enzyme (SCTE), an enzyme that cleaves the cathelicidin precursor protein, with serine protease inhibitors. There are a number of commercially and clinically relevant serine protease inhibitors that can be used in the methods and compositions of the disclosure. Thus, a composition and method useful for treatment of skin inflammation such as rosacea can comprise any number of serine protease inhibitors such as those disclosed in, for example, U.S. Pat. No. 5,786,328, U.S. Pat. No. 5,770,568, or U.S. Pat. No. 5,464,820, the disclosures of which are incorporated herein by reference.

In one aspect of the disclosure, a method of treatment of inflammatory diseases and disorders, rosacea and or acnes comprises inhibiting cathelicidin expression or activity. Examples of compositions and methods for inhibiting the expression of cathelicidins include antisense, ribozyme and gene therapy techniques. For example, rosacea can be inhibited or treated using antisense or ribozyme therapies that reduce the expression of cathelicidin. In one aspect, a vitamin D inhibitor, or vitamin D receptor antagonist can be used to reduce expression of a cathelicidin. Examples of compositions and methods for inhibiting cathelicidin activity include antibodies and small molecule agents. In one aspect, the treatment is at the site of inflammation through topical inhibition of Vitamin D activity, inhibiting of a Vitamin D receptor activity, or an inhibitor of a protease that cleaves full length cathelicidin into its active fragments is provided. For example, a method of the disclosure comprises inhibiting the kallikrein stratum corneum tryptic enzyme (SCTE), an enzyme that cleaves the cathelicidin precursor protein. Serine protease inhibitors such as aprotinin and 4-(2-aminoethyl)-benzenesulfonylfluoride (AEBSF) can inhibit this enzyme in vitro.

A vitamin D receptor inhibitor includes antagonistic vitamin D analogs, small molecules, and soluble vitamin D receptor polypeptides. For example, a class of vitamin D analogs referred to as 19-nor vitamin D analogs, which are characterized by the replacement of the A-ring exocyclic methylene group (carbon 19), typical of the vitamin D system, by two hydrogen atoms are useful for generating receptor antagonists. Further substitution at the 2-position and/or modification of the side chain attached to carbon 17 of the five-membered ring has led to pharmacologically active compounds at physiologically active concentrations compared to the native hormone. Related compounds having a 2α-methyl group have also been disclosed (Fujishima et al., Bioorg. Med. Chem. 11, 3621-3631, 2003). Select analogs exhibit antagonistic activity with respect to the vitamin D receptor and are effective for use in treating rosacea. Various methods of synthesizing 19-nor-vitamin D analogs have been disclosed (see, e.g., Perlman et al., Tetrahedron Lett. 31, 1823 (1990); Perlman et al., Tetrahedron Lett. 32, 7663 25 (1991), and DeLuca et al., U.S. Pat. No. 5,086,191). The synthesis of intermediates for use in the preparation of various 19-nor vitamin D analogs is disclosed in U.S. Pat. No. 5,086,191, which is incorporated by reference herein. Another antagonist includes 6-fluoro-vitamin D3 (6-F-D3). The disclosure provides methods for antagonizing the vitamin D receptor, methods for treating conditions such as rosacea, and the use of various vitamin D analogs in preparing medicaments for use in antagonizing the vitamin D receptor and/or treating conditions such as rosacea.

A inflammatory inhibitory composition (e.g., a rosacea inhibitory composition) of the disclosure used in the treatment of rosacea comprises (i) a cathelicidin activity or expression inhibitor, (ii) a serine protease activity or expression inhibitor (e.g., a SCTE inhibitor), or (ii) a combination of (i) and (ii). A cathelicidin activity inhibitor includes any agent that reduces the biological activity of a cathelicidin polypeptide (e.g., an N-terminal or C-terminal domain (e.g., LL37) of cathelicidin). Exemplary cathelicidin inhibitory agents include antibodies that bind to and inhibit a cathelicidin polypeptide or functional fragment thereof, enzymes that degrade cathelicidin polypeptide to inactive peptides and the like. A cathelicidin expression inhibitor includes, for example, antisense molecules, ribozymes and small molecule agents (e.g., vitamin D3 antagonists) that reduce the transcription or translation of a cathelicidin polynucleotide (e.g., DNA or RNA). A serine protease activity inhibitor includes any agent that reduces the biological activity of a serine protease polypeptide (e.g., a SCTE polypeptide). Exemplary serine protease inhibitory agents include antibodies that bind to and inhibit a serine protease polypeptide or functional fragment thereof, enzymes that degrade a serine protease polypeptide to inactive peptides, and the like. A serine protease expression inhibitor includes, for example, antisense molecules, ribozymes and small molecule agents (e.g., vitamin D antagonists) that reduce the transcription or translation of a serine protease polynucleotide (e.g., DNA or RNA).

In one aspect, an inflammatory/rosacea inhibitory composition of the disclosure may be formulated for topical administration (e.g., as a lotion, cream, spray, gel, or ointment). Such topical formulations are useful in treating or inhibiting rosacea at the site of the disorder. Examples of formulations in the market place include topical lotions, creams, soaps, wipes, and the like. It may be formulated into liposomes to reduce toxicity or increase bioavailability. Other methods for delivery of the composition include oral methods that entail encapsulation of the cathelicidin inhibitor in microspheres or proteinoids, aerosol delivery (e.g., to the lungs), or transdermal delivery (e.g., by iontophoresis or transdermal electroporation) and eye drops. Other methods of administration will be known to those skilled in the art.

Preparations for parenteral administration of an inflammatory/rosacea inhibitory composition of the disclosure include sterile aqueous or non-aqueous solutions, suspensions, and emulsions. Examples of non-aqueous solvents are propylene glycol, polyethylene glycol, vegetable oils (e.g., olive oil), and injectable organic esters such as ethyl oleate. Examples of aqueous carriers include water, saline, and buffered media, alcoholic/aqueous solutions, and emulsions or suspensions. Examples of parenteral vehicles include sodium chloride solution, Ringer's dextrose, dextrose and sodium chloride, lactated Ringer's, and fixed oils. Intravenous vehicles include fluid and nutrient replenishers, electrolyte replenishers (such as those based on Ringer's dextrose), and the like. Preservatives and other additives such as, other antimicrobial, anti-oxidants, cheating agents, inert gases and the like also can be included.

Typically an inflammatory/rosacea inhibitory composition of the disclosure will comprise a pharmaceutically acceptable carrier and may comprise one or more additional agents useful for delivery to a subject. An inflammatory/rosacea inhibitory composition will typically be formulated for topical application to a site of inflammatory or rosacea.

A pharmaceutical or cosmetic composition of the disclosure comprises, for example, an inflammatory/rosacea inhibitory composition and one or more additional agents. The one or more additional agents can include a pharmaceutically acceptable carrier alone or in combination with a skin lightening agent, a sunscreen agent, a skin conditioning agent, a skin protectant, an emollient, a humectant, or a mixture thereof. Various formulations for topical delivery are known in the art.

Suitable skin lightening agents include, but are not limited to, ascorbic acid and derivatives thereof; kojic acid and derivatives thereof; hydroquinone; azelaic acid; and various plant extracts, such as those from licorice, grape seed, and bear berry. A skin conditioning agent includes, for example, a substance that enhances the appearance of dry or damaged skin, as well as a material that adheres to the skin to reduce flaking, restore suppleness, and generally improve the appearance of skin. Representative examples of a skin conditioning agent that may be used include: acetyl cysteine, N-acetyl dihydrosphingosine, acrylates/behenyl acrylate/dimethicone acrylate copolymer, adenosine, adenosine cyclic phosphate, adenosine phosphate, adenosine triphosphate, alanine, albumen, algae extract, allantoin and derivatives, aloe barbadensis extracts, amyloglucosidase, arbutin, arginine, bromelain, buttermilk powder, butylene glycol, calcium gluconate, carbocysteine, carnosine, beta-carotene, casein, catalase, cephalins, ceramides, chamomilla recutita (matricaria) flower extract, cholecalciferol, cholesteryl esters, coco-betaine, corn starch modified, crystallins, cycloethoxymethicone, cysteine DNA, cytochrome C, darutoside, dextran sulfate, dimethicone copolyols, dimethylsilanol hyaluronate, elastin, elastin amino acids, ergocalciferol, ergosterol, fibronectin, folic acid, gelatin, gliadin, beta-glucan, glucose, glycine, glycogen, glycolipids, glycoproteins, glycosaminoglycans, glycosphingolipids, horseradish peroxidase, hydrogenated proteins, hydrolyzed proteins, jojoba oil, keratin, keratin amino acids, and kinetin. Other non-limiting examples of a skin conditioning agent that may be included in the compositions includes lactoferrin, lanosterol, lecithin, linoleic acid, linolenic acid, lipase, lysine, lysozyme, malt extract, maltodextrin, melanin, methionine, niacin, niacinamide, oat amino acids, oryzanol, palmitoyl hydrolyzed proteins, pancreatin, papain, polyethylene glycol, pepsin, phospholipids, phytosterols, placental enzymes, placental lipids, pyridoxal 5-phosphate, quercetin, resorcinol acetate, riboflavin, saccharomyces lysate extract, silk amino acids, sphingolipids, stearamidopropyl betaine, stearyl palmitate, tocopherol, tocopheryl acetate, tocopheryl linoleate, ubiquinone, vitis vinifera (grape) seed oil, wheat amino acids, xanthan gum, and zinc gluconate. Skin protectant agents include, for example, a compound that protects injured or exposed skin or mucous membrane surfaces from harmful or irritating external compounds. Representative examples include algae extract, allantoin, aluminum hydroxide, aluminum sulfate, camellia sinensis leaf extract, cerebrosides, dimethicone, glucuronolactone, glycerin, kaolin, lanolin, malt extract, mineral oil, petrolatum, potassium gluconate, and talc.

An emollient may be included in a pharmaceutical or cosmetic composition of the disclosure. An emollient generally refers to a cosmetic ingredient that can help skin maintain a soft, smooth, and pliable appearance. Emollients typically remain on the skin surface, or in the stratum corneum, to act as a lubricant and reduce flaking. Some examples of an emollient include acetyl arginine, acetylated lanolin, algae extract, apricot kernel oil polyethylene glycol-6 esters, avocado oil polyethylene glycol-11 esters, bis-polyethylene glycol-4 dimethicone, butoxyethyl stearate, glycol esters, alkyl lactates, caprylyl glycol, cetyl esters, cetyl laurate, coconut oil polyethylene glycol-10 esters, alkyl tartrates, diethyl sebacate, dihydrocholesteryl butyrate, dimethiconol, dimyristyl tartrate, disteareth-5 lauroyl glutamate, ethyl avocadate, ethylhexyl myristate, glyceryl isostearates, glyceryl oleate, hexyldecyl stearate, hexyl isostearate, hydrogenated palm glycerides, hydrogenated soy glycerides, hydrogenated tallow glycerides, isostearyl neopentanoate, isostearyl palmitate, isotridecyl isononanoate, laureth-2 acetate, lauryl polyglyceryl-6 cetearyl glycol ether, methyl gluceth-20 benzoate, mineral oil, myreth-3 palmitate, octyldecanol, octyldodecanol, odontella aurita oil, 2-oleamido-1,3 octadecanediol, palm glycerides, polyethylene glycol avocado glycerides, polyethylene glycol castor oil, polyethylene glycol-22/dodecyl glycol copolymer, polyethylene glycol shea butter glycerides, phytol, raffinose, stearyl citrate, sunflower seed oil glycerides, and tocopheryl glucoside.

Humectants are cosmetic ingredients that help maintain moisture levels in skin. Examples of humectants include acetyl arginine, algae extract, aloe barbadensis leaf extract, 2,3-butanediol, chitosan lauroyl glycinate, diglycereth-7 malate, diglycerin, diglycol guanidine succinate, erythritol, fructose, glucose, glycerin, honey, hydrolyzed wheat protein/polyethylene glycol-20 acetate copolymer, hydroxypropyltrimonium hyaluronate, inositol, lactitol, maltitol, maltose, mannitol, mannose, methoxy polyethylene glycol, myristamidobutyl guanidine acetate, polyglyceryl sorbitol, potassium pyrollidone carboxylic acid (PCA), propylene glycol, sodium pyrollidone carboxylic acid (PCA), sorbitol, sucrose, and urea.

A pharmaceutical or cosmetic composition of the disclosure comprises, for example, an inflammatory/rosacea inhibitory composition and a fatty alcohol, a fatty acid, an organic base, an inorganic base, a preserving agent, a wax ester, a steroid alcohol, a triglyceride ester, a phospholipid, a polyhydric alcohol ester, a fatty alcohol ether, a hydrophilic lanolin derivative, a hydrophilic beeswax derivative, a cocoa butter wax, a silicon oil, a pH balancer, a cellulose derivative, a hydrocarbon oil, or a mixture thereof. Non-limiting examples of a suitable phospholipid include lecithin and cephalin. Suitable hydrocarbon oils include, but are not limited to, palm oil, coconut oil, and mineral oil. Additional ingredients may be included in the above compositions to vary the texture, viscosity, color and/or appearance thereof, as is appreciated by one of ordinary skill in the art.

A pharmaceutical or cosmetic composition of the disclosure can be formulated as an emulsion. Either a water-in-oil or oil-in-water emulsion may be formulated. Examples of suitable surfactants and emulsifying agents include nonionic ethoxylated and nonethoxylated surfactants, abietic acid, almond oil polyethylene glycol, beeswax, butylglucoside caprate, glycol ester, alkyl phosphate, caprylic/capric triglyceride polyethylene glycol4 esters, ceteareth-7, cetyl alcohol, cetyl phosphate, corn oil polyethylene glycol esters, dextrin laurate, dilaureth-7 citrate, dimyristyl phosphate, glycereth-17 cocoate, glyceryl erucate, glyceryl laurate, hydrogenated castor oil polyethylene glycol esters, isosteareth-11 carboxylic acid, lecithin, lysolecithin, nonoxynol-9, octyldodeceth-20, palm glyceride, polyethylene glycol diisostearate, polyethylene glycol stearamine, poloxamines, potassium linoleate, raffinose myristate, sodium caproyl lactylate, sodium caprylate, sodium cocoate, sodium isostearate, sodium tocopheryl phosphate, steareths, and trideceths. Thickening agents suitable for inclusion in a composition or formulation herein include those agents commonly used in skin care preparations. More specifically, such examples include acrylamides copolymer, agarose, amylopectin, bentonite, calcium alginate, calcium carboxymethyl cellulose, carbomer, carboxymethyl chitin, cellulose gum, dextrin, gelatin, hydrogenated tallow, hydroxyethylcellulose, hydroxypropylcellulose, hydroxpropyl starch, magnesium alginate, methylcellulose, microcrystalline cellulose, pectin, various polyethylene glycol's, polyacrylic acid, polymethacrylic acid, polyvinyl alcohol, various polypropylene glycols, sodium acrylates copolymer, sodium carrageenan, xanthan gum, and yeast beta-glucan.

The disclosure also includes methods that utilize a composition described herein to treat rosacea comprising contacting the skin with a composition of the disclosure. The compositions are typically applied topically to human skin. Accordingly, such a composition is formulated, in a further embodiment, as a liquid, cream, gel, oil, fluid cream or milk, lotion, emulsion, or microemulsion. In a related embodiment, the composition further comprises an excipient adapted for application to the face and neck. Such an excipient should have a high affinity for the skin, be well tolerated, stable, and yield a consistency that allows for easy and pleasant utilization.

The term “contacting” refers to exposing a cell or subject to a rosacea inhibitor composition such that cathelicidin production or expression is inhibited or reduced or proteases necessary for activation of cathelicidin to produce LL-37 are inhibited or reduced. Contacting can occur in vivo, for example by administering the composition to a subject afflicted with a rosacea. In vivo contacting includes both parenteral as well as topical. “Inhibiting” or “inhibiting effective amount” refers to the amount of an inflammatory/rosacea inhibitory composition that is sufficient to cause, for example, a decrease in cathelicidin production or activity, protease production or activity, or a reduction in symptoms associated with rosacea (e.g., preventing or ameliorating a sign or symptoms of a disorder such as a rash, sore, and the like) as compared to a control subject or sample.

In one aspect of the disclosure, cathelicidin inhibitor is contacted with a subject to inhibit/reduce host cell defense mechanisms by, for example, inhibiting or reducing cathelicidin production and processing.

Any of a variety of art-known methods can be used to administer a cathelicidin inhibitor to a subject. For example, the cathelicidin inhibitor of the disclosure can be administered parenterally by injection or by gradual infusion over time. The composition can be administered intravenously, intraperitoneally, intramuscularly, subcutaneously, intracavity, or transdermally.

Generally, the optimal dosage of the inflammatory/rosacea inhibitory composition will depend upon the disorder and factors such as the weight of the subject, the type and severity of rosacea, the weight, sex, and degree of symptoms. Nonetheless, suitable dosages can readily be determined by one skilled in the art.

An amount of a composition effective to treat rosacea is used in the methods of the disclosure. For example, a small amount of the composition (from about 0.1 ml to about 5 ml) is applied to exposed areas of affected skin from a suitable container or applicator, and, if necessary, the composition is then spread over and/or rubbed into the skin using the hand, finger, or other suitable device. A composition disclosed herein is typically packaged in a container that is appropriate in view of its viscosity and intended use by a subject. For example, a lotion or fluid cream may be packaged in a bottle, roll-ball applicator, capsule, propellant-driven aerosol device, or a container fitted with a manually operated pump. A cream may simply be stored in a non-deformable bottle, or in a squeeze container, such as a tube or a lidded jar.

If desired, a suitable therapy regime can combine administration of an inflammatory/rosacea inhibitory composition of the disclosure with one or more additional therapeutic agents (e.g., an inhibitor of TNF, an antibiotic, and the like). For example, advising the patient to avoid those stimuli that tend to exacerbate the disease—exposure to extremes of heat and cold, excessive sunlight, ingestion of hot liquids, alcohol, and spicy foods—may help. Although its mechanism of action is not clearly understood, the mainstay of treatment is the use of oral tetracycline, especially for the papular or pustular lesions. The dosage utilized is generally 250 mg every 6 hours for the first 3 to 4 weeks, followed by tapering based on clinical response. Doxycycline and minocycline (50-100 mg every 12 hours) are also effective and have the advantage of less frequent dosage and less concern over problems with gastrointestinal absorption. Patients who are intolerant to the tetracyclines may benefit from the use of erythromycin. Oral isotretinoin, in doses similar to those used for acne vulgaris, has also been effective for the inflammatory lesions, erythema, and rhinophyma. Other oral agents that have been used include ampicillin and metronidazole. Clonidine may also be of some value in reducing facial flushing. Topical therapy for rosacea is generally less successful than systemic treatment, although often tried first. Metronidazole (2-methyl-5-nitroimidazole-1-ethanol) may be effective topically; it is available commercially as a 0.75% gel and, when applied twice daily, substantially reduces inflammatory lesions; it is classified as an antiprotozoal. Although topical corticosteroid may effectively improve signs and symptoms, long-term therapy is not advisable since it may cause atrophy, chronic vasodilation, and telangiectasia formation. The treatment of chronic skin changes may require surgical intervention.

Serine protease inhibitors are shown herein to play a major role in the direct inactivation of the mediators of inflammation. A cocktail of serine protease inhibitors, their analogs, salts or derivatives, appears to provide treatment when used in combination with a corticosteroid.

The typical course of antimicrobial treatment is to start with metronidazole, and if that is not as effective as desired to ameliorate the symptoms, or the condition worsens, then therapy is switched to a stronger antimicrobial, such as tetracycline or minocycline. This standard course of therapy persists under the pretense that the antimicrobial is reducing inflammation, because inflammation appears to be reduced, even though it is logically the antimicrobial effects that cause the reduction in inflammation (and because these types of compounds are not known to have anti-inflammatory properties).

The inflammatory/rosacea inhibitory composition(s), other therapeutic agents, and/or antibiotic(s) can be administered, simultaneously, but may also be administered sequentially.

Suitable antibiotics include aminoglycosides (e.g., gentamicin), beta-lactams (e.g., penicillins and cephalosporins), quinolones (e.g., ciprofloxacin), and novobiocin.

The disclosure also provides a method of diagnosing rosacea. In one aspect, the method comprises identifying higher levels of cathelicidin in the skin of a subject, or the processed form of cathelicidin (LL-37 or FA-29) in a subject suspected of having rosacea. In another aspect, the method of diagnoses further includes measuring the activity or expression of serine proteases. In yet a further aspect, the serine protease is kallikrein stratum corneum tryptic enzyme (SCTE), variant or homolog thereof. In yet another aspect, the disclosure includes measuring the protein levels in a sample from a subject suspected of having rosacea and measuring the level of protein, wherein an increased level of protein compared to a control is indicate of rosacea or a risk of having rosacea. For example, the level of cathelicidin is increased in a subject having rosacea, therefore an increased protein concentration will coincide with an increased risk of rosacea. Currently, rosacea is diagnosed by clinical symptoms and can be confused with other dermatological diseases such as acne. Accordingly, the methods of compositions of the disclosure can be used in distinguishing rosacea from other dermatological or autoimmune diseases.

Sample and measurements of cathelicidin, serine protease or a combination of both can be performed in any number of methods known in the art. In one example, the method includes measuring a panel of biomarkers comprising a cathelicidin and a serine protease. A biomarker refers to a detectable biological entity associated with a particular phenotype or risk of developing a particular phenotype. The biological entity can be a polypeptide or polynucleotide. A biomarker to be detected is referred to as a target. For example, a target polynucleotide refers to a biomarker comprising a polynucleotide (e.g., an mRNA or cDNA) that is to be detected. In another example, a target polypeptide refers to a protein expressed (i.e., transcribed and translated) that is to be detected. A biomarker, as defined by the National Institutes of Health (N1H), refers to a molecular indicator of a specific biological property; a biochemical feature or facet that can be used to measure the progress of disease or the effects of treatment. A panel of biomarkers is a selection of at least two biomarkers. Biomarkers may be from a variety of classes of molecules. Thus, a biomarker panel of the disclosure comprises a cathelicidin polypeptide or polynucleotide and a serine protease (e.g. a kallikrein) polypeptide or polynucleotide. Other biomarkers can be used in the compositions and methods of the disclosure such as, but not limited to, inflammatory biomarkers (e.g., cytokines) and the like.

Panels comprising a polypeptide or polynucleotide can be generated using methods known in the art including, but not limited to, ELISA techniques, nucleic acid chips (e.g., DNA chips). Oligonucleotide for use in nucleic acid panels can be identified and generated based upon sequence for cathelicidins, serine proteases, and cytokines available to one of skill in the art.

Polypeptides can be used in the generation of antibodies that can be used in the method and compositions of the disclosure. For example, antibodies directed to cathelicidins, other antimicrobial peptides (AMPs), serine proteases (e.g., kallikrein) and the like, can be generated or commercially obtained. Such antibodies can be used in the method described herein.

Any of the oligonucleotides or nucleic acids of the disclosure can be labeled by incorporating a detectable label measurable by spectroscopic, photochemical, biochemical, immunochemical, or chemical means. For example, such labels can comprise radioactive substances (e.g., ³²P, ³⁵S, ³H, ¹²⁵I), fluorescent dyes (e.g., 5-bromodesoxyuridin, fluorescein, acetylaminofluorene, digoxigenin), biotin, nanoparticles, and the like. Such oligonucleotides are typically labeled at their 3′ and 5′ ends.

A probe refers to a molecule which can detectably distinguish changes in gene expression or can distinguish between target molecules differing in structure. Detection can be accomplished in a variety of different ways depending on the type of probe used and the type of target molecule. Thus, for example, detection may be based on discrimination of activity levels of the target molecule, but typically is based on detection of specific binding. Examples of such specific binding include antibody binding and nucleic acid probe hybridization. Thus, for example, probes can include enzyme substrates, antibodies and antibody fragments, and nucleic acid hybridization probes (including primers useful for polynucleotide amplification and/or detection). Thus, in one embodiment, the detection of the presence or absence of the at least one target polynucleotide involves contacting a biological sample with a probe, typically an oligonucleotide probe, where the probe hybridizes with a form of a target polynucleotide in the biological sample containing a complementary sequence, where the hybridization is carried out under selective hybridization conditions. Such an oligonucleotide probe can include one or more nucleic acid analogs, labels or other substituents or moieties so long as the base-pairing function is retained.

A reference or control population refers to a group of subjects or individuals who are predicted to be representative of the genetic variation found in the general population having a particular genotype or expression profile. Typically, the reference population represents the genetic variation in the population at a certainty level of at least 85%, typically at least 90%, least 95% and but commonly at least 99%. The reference or control population can include subjects who individually have not demonstrated any disease or disorder of the skin (e.g., rosacea) and can include individuals whose family line does not or has not demonstrated any skin diseases or disorders.

For example, diagnosis can be performed by quantitative immunoblot of cathelicidin and/or a serine protease (e.g., SCTE) from tape-stripped skin or by mass spectrometry. The level of expressed polypeptide can be measured by ELIZA techniques, by immunoblot, by polypeptide-based microfluidic techniques, by electrochemical or resistometric sensors and the like. Alternatively, the level of nucleic acid encoding a cathelicidin and/or serine protease, such as SCTE, can be measured. Method of nucleic acid measurement include northern blot techniques, PCR, nucleic acid chip-based assays, and the like.

A tape stripping method typically involves applying an adhesive tape to the skin of a subject and removing the adhesive tape from the skin of the subject one or more times. In certain examples, the adhesive tape is applied to the skin and removed from the skin about one to ten times. Alternatively, about ten adhesive tapes can be applied to the skin and removed from the skin. These adhesive tapes are then combined for further analysis.

A substrate comprising a plurality of oligonucleotide primers or probes of the disclosure may be used either for detecting or amplifying targeted sequences. The oligonucleotide probes and primers of the disclosure can be attached in contiguous regions or at random locations on the solid support. Alternatively the oligonucleotides of the disclosure may be attached in an ordered array wherein each oligonucleotide is attached to a distinct region of the solid support which does not overlap with the attachment site of any other oligonucleotide. Typically, such oligonucleotide arrays are “addressable” such that distinct locations are recorded and can be accessed as part of an assay procedure. The knowledge of the location of oligonucleotides on an array make “addressable” arrays useful in hybridization assays. For example, the oligonucleotide probes can be used in an oligonucleotide chip such as those marketed by Affymetrix and described in U.S. Pat. No. 5,143,854; PCT publications WO 90/15070 and 92/10092, the disclosures of which are incorporated herein by reference. These arrays can be produced using mechanical synthesis methods or light directed synthesis methods which incorporate a combination of photolithographic methods and solid phase oligonucleotide synthesis.

The immobilization of arrays of oligonucleotides on solid supports has been rendered possible by the development of a technology generally referred to as “Very Large Scale Immobilized Polymer Synthesis” in which probes are immobilized in a high density array on a solid surface of a chip (see, e.g., U.S. Pat. Nos. 5,143,854; and 5,412,087 and in PCT Publications WO 90/15070, WO 92/10092 and WO 95/11995, each of which are incorporated herein by reference), which describe methods for forming oligonucleotide arrays through techniques such as light-directed synthesis techniques.

In another aspect, an array of oligonucleotides complementary to subsequences of the target gene is used to determine the identity of the target, measure its amount, and detect differences between the target and a reference wild-type sequence.

Hybridization techniques can also be used to identify the biomarkers of the disclosure and thereby determine a predictive skin disease or disorder. In this aspect, expression profiles or polymorphism(s) are identified based upon the higher thermal stability of a perfectly matched probe compared to the mismatched probe. The hybridization reactions may be carried out in a solid support (e.g., membrane or chip) format, in which, for example, the target nucleic acids are immobilized on nitrocellulose or nylon membranes and probed with oligonucleotide probes of the disclosure. Any of the known hybridization formats may be used, including Southern blots, slot blots, “reverse” dot blots, solution hybridization, solid support based sandwich hybridization, bead-based, silicon chip-based and microtiter well-based hybridization formats.

Hybridization of an oligonucleotide probe to a target polynucleotide may be performed with both entities in solution, or such hybridization may be performed when either the oligonucleotide or the target polynucleotide is covalently or noncovalently affixed to a solid support. Attachment may be mediated, for example, by antibody-antigen interactions, poly-L-Lys, streptavidin or avidin-biotin, salt bridges, hydrophobic interactions, chemical linkages, UV cross-linking baking, etc. Oligonucleotides may be synthesized directly on the solid support or attached to the solid support subsequent to synthesis. Solid-supports suitable for use in detection methods of the disclosure include substrates made of silicon, glass, plastic, paper and the like, which may be formed, for example, into wells (as in 96-well plates), slides, sheets, membranes, fibers, chips, dishes, and beads. The solid support may be treated, coated or derivatized to facilitate the immobilization of the allele-specific oligonucleotide or target nucleic acid.

In one aspect, a sandwich hybridization assay comprises separating the variant and/or wild-type target nucleic acid biomarker in a sample using a common capture oligonucleotide immobilized on a solid support and then contact with specific probes useful for detecting the variant and wild-type nucleic acids. The oligonucleotide probes are typically tagged with a detectable label.

Hybridization assays based on oligonucleotide arrays rely on the differences in hybridization stability of short oligonucleotides to perfectly matched and mismatched target variants. Efficient access to expression or polymorphic information is obtained through a basic structure comprising high-density arrays of oligonucleotide probes attached to a solid support (the chip) at selected positions. Each DNA chip can contain thousands to millions of individual synthetic DNA probes arranged in a grid-like pattern and miniaturized to the size of a dime or smaller. Such a chip may comprise oligonucleotides representative of both a wild-type and variant sequences.

Oligonucleotides of the disclosure can be designed to specifically hybridize to a target region of a polynucleotide. As used herein, specific hybridization means the oligonucleotide forms an anti-parallel double-stranded structure with the target region under certain hybridizing conditions, while failing to form such a structure when incubated with a different target polynucleotide or another region in the polynucleotide or with a polynucleotide lacking the desired locus under the same hybridizing conditions. Typically, the oligonucleotide specifically hybridizes to the target region under conventional high stringency conditions.

A nucleic acid molecule such as an oligonucleotide or polynucleotide is said to be a “perfect” or “complete” complement of another nucleic acid molecule if every nucleotide of one of the molecules is complementary to the nucleotide at the corresponding position of the other molecule. A nucleic acid molecule is “substantially complementary” to another molecule if it hybridizes to that molecule with sufficient stability to remain in a duplex form under conventional low-stringency conditions. Conventional hybridization conditions are described, for example, in Sambrook et al., Molecular Cloning, A Laboratory Manual, 2nd ed., Cold Spring Harbor Press, Cold Spring Harbor, N.Y. (1989), and in Haymes et al., Nucleic Acid Hybridization, A Practical Approach, IRL Press, Washington, D.C. (1985). While perfectly complementary oligonucleotides are used in most assays for detecting target polynucleotides or polymorphisms, departures from complete complementarity are contemplated where such departures do not prevent the molecule from specifically hybridizing to the target region. For example, an oligonucleotide primer may have a non-complementary fragment at its 5′ or 3′ end, with the remainder of the primer being complementary to the target region. Those of skill in the art are familiar with parameters that affect hybridization; such as temperature, probe or primer length and composition, buffer composition and salt concentration and can readily adjust these parameters to achieve specific hybridization of a nucleic acid to a target sequence.

A variety of hybridization conditions may be used in the disclosure, including high, moderate and low stringency conditions; see for example Maniatis et al., Molecular Cloning: A Laboratory Manual, 2d Edition, 1989, and Short Protocols in Molecular Biology, ed. Ausubel, et al., hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength and pH. The Tₘ is the temperature (under defined ionic strength, pH and nucleic acid concentration) at which 50% of the probes complementary to the target hybridize to the polyadenylated mRNA target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of helix destabilizing agents such as formamide. The hybridization conditions may also vary when a non-ionic backbone, i.e., PNA is used, as is known in the art. In addition, cross-linking agents may be added after target binding to cross-link, i.e., covalently attach, the two strands of the hybridization complex.

The same strip of tape can be repeatedly applied to, and removed from, a target site, such as a rosacea site or area suspected of comprising rosacea. However, a fresh piece of adhesive tape is sequentially applied to a target site of the skin. The individual tape strips used to sample a site can then be combined into one extraction vessel for further processing such as protein extraction or nucleic acid extraction.

Factors such as the flexibility, softness, and composition of the adhesive tape used, the time the tape is allowed to adhere to the skin before it is removed, the force applied to the tape as it is applied to the skin, the prevalence of a biological product (e.g., protein or nucleic acid) being analyzed, the disease status of the skin, and subject variability are typically taken into account in deciding on a protocol useful for a particular tape stripping method to assure that sufficient sample is obtained. Tape stripping is stopped before viable epidermis is exposed by ceasing tape stripping before the tissue glistens. Therefore, the tape stripping method is considered a “noninvasive” method.

Biological factors can be isolated from the tape strips by methods known in the art. Such biological factors includes cells, polypeptide, and polynucleotides. The isolated biological factors can be used in the methods described herein for the detection and diagnosis or rosacea.

The following examples are intended to illustrate but not limit the disclosure. While they are typical of those that might be used, other procedures known to those skilled in the art may alternatively be used.

### EXAMPLES

### Materials and Methods

Immunohistochemistry. 3 mm punch biopsies were taken from untreated lesional skin of rosacea and normal volunteers. Skin was frozen in Tissue-Tek™.

O.C.T. (Electron Microscopy Sciences, Fort Wash., Pa.) and 5 μm sections cut and fixed in methanol for 30 min at 4° C., blocked with 5% donkey serum in phosphate buffered saline (PBS) for 30 min, incubated 1 hr with polyclonal chicken anti-hCAP18/LL-37 antibody, then incubated 30 min with goat anti-chicken HRP antibody and developed using the Vectastain ABC kit (Vecta Laboratory Inc., Burlingame, Calif.). Images were obtained using an Olympus BX41 microscope (Scientific Instrument Company, Temecula, Calif.). Rosacea samples and 10 normal were examined and scored by a blinded investigator.

Cathelicidin Protein analysis. Facial skin was tape-stripped 20 times with 23 mm diameter tape (D-Squame™, CuDerm Corp., Dallas, Tex.). The tapes were immersed in 1 ml of 1 M HCl, 1% TFA and vortexed. Extracts were lyophilized then pellet dissolved in 100 μl of distilled water and protein measured using the BCA protein assay (Pierce Biotechnology, Inc., Rockford Ill.). Cathelicidin was measured by quantitative immunoblot.

Surface Enhanced Laser Desorption/Ionization Time-of-Flight Mass Spectrometry (SELDI-TOF-MS). Frozen skin in OCT was sectioned into twenty 10-μm slices and dissolved in 100 μl of RIPA buffer (50 mM HEPES, 150 mM NaCl, 0.05% SDS, 0.25% deoxycholate, 0.5% NP-40, pH 7.4) containing protease inhibitors (Roche Applied Science). Samples were sonicated for 3 min and centrifuged for 10 min at 14,000 rpm. Supernatant was then applied to protein chips (RS-100, Ciphergen Biosystems, Fremont, Calif.) previously coated with 4 μl of anti-LL-37 rabbit antibody for 2 h at RT, and blocked with 0.5 M ethanolamine in PBS (pH 8.0). After washing three times with PBS/0.5% triton X, protein chips were assembled in the Bioprocessor™ reservoir and fifty μl of eluate applied for 2 h at RT. Protein chips were washed twice with RIPA buffer, once with PBS/0.5% triton X, and three times with PBS, followed by soaking in 10 mM HEPES buffer, then air-dried. 0.5 μl of energy absorbance material (50%-saturated alpha-cyano-4-hydroxy cinnamic acid in 50% acetonitrile, 0.5% trifluoric acid) was applied twice, then dried. Samples were analyzed on a SELDI mass analyzer PBS II (Ciphergen Biosystems) using time-lag focusing. Specificity and calibration was confirmed by synthetic cathelicidin peptides LL-37 and KR-20 used as internal references.

Fluorescence immunohistochemistry. Frozen sections (6 μm) were fixed with paraformaldehyde, blocked with 5% goat serum and incubated with polyclonal rabbit anti-LL-37 or monoclonal mouse anti-stratum corneum tryptic enzyme (SCTE). Goat anti-rabbit IgG conjugated to FITC and goat anti-mouse IgG conjugated to AlexaFluor568 (Molecular Probes, Eugene, Oreg.) were used as secondary antibodies, respectively. Sections were mounted in ProLong Anti-Fade reagent (Molecular Probes). Images were obtained using a Zeiss LSM510 laser scanning confocal microscope coupled with an Axiovert 100 inverted stage microscope.

In situ zymography. Frozen sections (6 μm) were rinsed with 1% tween-20 in dH2O and incubated with 100 μl of BODIPY-FL-casein substrate (10 μg/ml in 10 mM Tris-HCl, pH 7.8, Molecular Probes, Inc., Eugene, Oreg.) at 37° C. for 3 h. After removal of excess of substrate solution, nuclei were stained with 4′,6-Diamidino-2-phenylindole (DAPI) and rinsed with 1% tween-20 in dH2O. Sections were mounted in ProLong Anti-Fade reagent (Molecular Probes). Images were obtained using an Olympus BX41 microscope (Scientific Instrument Company, Temecula, Calif.).

Skin protease activity. To obtain skin surface protease, facial skin was tape-stripped 20 times then immersed in 1 ml 1 M acetic acid and incubated 4° C. overnight, extracts were lyophilized and the pellet dissolved in 40 μl of 1×PBS, pH 7.4. Protease activity was monitored with EnzCheck® Protease Assay Kit green fluorescence (Molecular Probes, Inc., Eugene, Oreg.) according to manufacturer's instructions. Briefly, 10 μl of the aqueous solution collected from the skin surface was mixed with 190 μl of BODIPY FL casein substrate in 10 mM Tris-HCl, pH 7.8, and incubated at 37° C. for 24 h. Protease activity was monitored as increased fluorescence with SpectraMax GEMINI EM (Molecular Devices Corporation, Sunnyvale, Calif.). In some experiments protease inhibitors were added including mixed protease inhibitors (Complete EDTA-free, 1 tablet/50 ml; Roche, Indianapolis, Ind.), 200 μg/ml bestatin, 20 μg/ml E-64, and 20 μg/ml aprotinin (Sigma-Aldrich, St. Louis, Mo.), 200 μM 4-(2-aminoethyl)-benzenesulfonylfluoride (AEBSF), 200 μM human neutrophil elastase inhibitor (methoxysuccinyl-Ala-Ala-Pro-Ala-chloromethyl ketone), 200 μM human leukocyte elastase inhibitor (methoxysuccinyl-Ala-Ala-Pro-Val-chloromethylketone, Calbiochem, San Diego, Calif.).

Peptide synthesis. Cathelicidin peptides were commercially prepared by Synpep (Dublin, Oreg.). Peptide amino acid sequences were LLGDFFRKSKEKIGKEFKRIVQRIKDFLRNLVPRTES (LL-37; SEQ ID NO:14), FALLGDFFRKSKEKIGKEFKRIVQRIKDF (FA-29; SEQ ID NO:10), DISCDKDNKRFALLGDFFRKSKEKIGK (DI-27; SEQ ID NO:7), and KRIVQRIKDFLRNLVPRTES (KR-20; SEQ ID NO:15). All synthetic peptides were purified to greater than 95% purity by HPLC, and identity was confirmed by mass spectrometry.

Measurement of IL-8 release. Normal human keratinocytes (Cascade Biologics) were grown in EpiLife medium (Cascade Biologics) supplemented with 0.06 mM Ca2, 1% EpiLife defined growth supplement, and 1% penicillin/streptomycin (Invitrogen Life Technologies). Cells were grown at 37° C. in a humidified atmosphere of 5% CO₂ and 95% air. Human keratinocytes were cultured to confluence and treated with the cathelicidin peptides (3.2 μM) for 6 h. Supernatants were collected and placed in a sterile 96-well plate for ELISA. IL-8 production was determined by ELISA (R&D systems) according to the manufacturer's instructions.

Mouse skin inflammation models. All animal procedures were approved by the Veterans Affairs (VA) San Diego Healthcare System subcommittee on animal studies. Balb/C and C57/B16 mice shaved 24 h prior to treatments and injected subcutaneously on the back with 40 μl of peptide (320 μM) twice a day. Forty-eight h after initial injection (total 4 injections), skin inflammation was assessed by the severity of erythema and edema, then biopsied for histology.

To determine the role of loss of cathelicidin in inflammation, mCRAMP-deficient (Cnlp^{-/-}) mice and wild-type Cnlp^{+/+} littermates were used. To induce cathelicidin, mice were shaved and skin lightly abraded 20 times with sandpaper (Aluminum oxide sandpaper, medium 100 grit, 3M, St. Paul, Minn.). Twenty-four h later, chemical inflammation was induced epicutaneously by application of 10 μl of 2% 2,4-dinitrofluorobenzene (DNFB, Sigma-Aldrich, St. Louis, Minn.) diluted in acetone onto abraded back skin. Skin was excised 5 days after the application of DFNB and fixed in 10% formaldehyde solution and processed for histology.

Surface Enhanced Laser Desorption/Ionization Time-of-Flight Mass Spectrometry (SELDI-TOF-MS). Skin biopsies were frozen in OCT compound, and stored at −80° C. Twenty 10-μm slices were collected in 1.5 ml polypropylene tubes and dissolved in 100 μl of RIPA buffer (50 mM HEPES, 150 mM NaCl, 0.05% SDS, 0.25% deoxycholate, 0.5% NP-40, pH 7.4) containing protease inhibitors (Roche Applied Science). Samples were sonicated for 3 min and centrifuged for 10 min at 14,000 rpm. Supernatant of RIPA buffer were transferred into new tubes and kept at −20° C. until SELDI-TOF analysis.

Protein chips (RS-100 protein chip array, Ciphergen Biosystems, Fremont, Calif.) were coated with 4 μl of anti-LL-37 rabbit antibody for 2 h at RT, followed by blocking with 0.5 M ethanolamine in PBS (pH 8.0). After washing three times with PBS/0.5% triton X, protein chips were assembled in the Bioprocessor™ reservoir and fifty upl of eluted samples were applied and incubated 2 h at RT. Protein chips were washed twice with RIPA buffer, once with PBS/0.5% tritonX, and three times with PBS, followed by soaking in 10 mM HEPES buffer, and air-dried. Half μl of energy absorbance molecule (50%-saturated alpha-cyano-4-hydroxy cinnamic acid in 50% acetonitrile, 0.5% trifluoric acid) was applied twice, and all spots were completely dried. Samples were analyzed on a SELDI mass analyzer PBS II with a linear time-of-flight mass spectrometer (Ciphergen Biosystems) using time-lag focusing. Specificity of anti-LL-37 antibody in this system was confirmed by several synthetic cathelicidin peptides. Synthetic LL-37 and KR-20 peptides were used as internal references to calibrate the exact mass sizes. Peptide sequences of each peaks were deduced from molecular mass size using FindPept tool in ExPASy (Expert Protein Analysis System) web site (http://www.expasy.org/).

Fluorescence immunohistochemistry. Frozen sections (6 μm) were fixed with paraformaldehyde, blocked with 5% goat serum and incubated with polyclonal rabbit anti-LL-37 or monoclonal mouse anti-stratum corneum tryptic enzyme (SCTE) primary antibodies. Goat anti-rabbit IgG conjugated to AlexaFluor 568 goat anti-mouse IgG (Molecular Probes, Eugene, Oreg.) conjugated to tetramethylrhodamine isothiocyanate (TRITC) were used as secondary antibodies, respectively. Sections were mounted in ProLong Anti-Fade reagent (Molecular Probes). Images were obtained using a Zeiss LSM510 laser scanning confocal microscope coupled with an Axiovert 100 inverted stage microscope.

Mouse skin inflammation models. Balb/C or C56/B16 mice shaved 24 h prior to treatments were injected subcutaneously on the back with 40 μl of peptide at different concentrations (320, 32, 3.2, 0.32 μM, and PBS as 0 μM) twice a day. Forty-eight h after initial injection (total 4 injection), skin inflammation was assessed by the severity of erythema and edema. Skin was then biopsied for hematoxlin eosin staining to examine the histopathological changes. Frozen sections were also prepared and processed for immunostaining that included rat monoclonal anti-mouse Ly-6G (BD Biosciences, San Jose, Calif.) or rat monoclonal anti-mouse CD31 (BD Biosciences). Goat anti-rat IgG conjugated to FITC (Abcam Inc., Cambridge, Mass.) was used as the secondary antibody. Nuclei were stained with DAPI and sections were mounted in ProLong Anti-Fade reagent (Molecular Probes). Images were obtained using an Olympus BX41 microscope (Scientific Instrument Company, Temecula, Calif.).

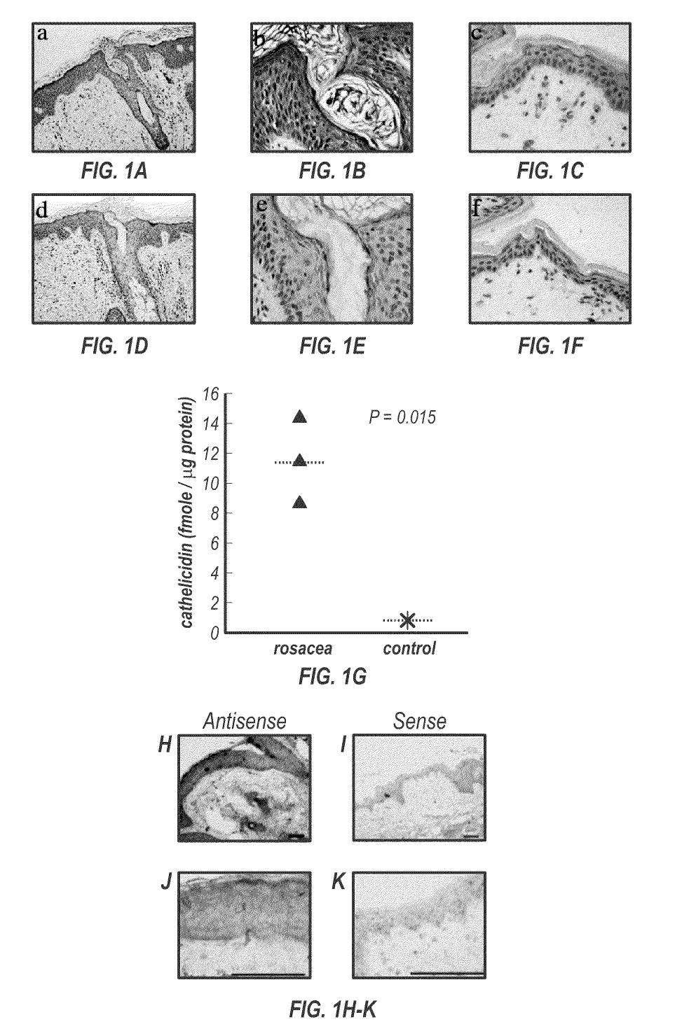

To test if the expression of cathelicidin is altered rosacea, skin biopsies were obtained from the naso-malar fold of rosacea patients and compared to skin from a similar location in normal individuals. All specimens from rosacea showed abundant cathelicidin by immunostaining, while normal facial skin showed minimal expression (FIG. 1a-f). Cathelicidin was abundant throughout the epidermis, but was not seen in healthy volunteers (FIG. 1e). To quantify this, epidermal cathelicidin was measured in tape-stripped samples. These had significantly higher cathelicidin in rosacea than in normal (FIG. 1g). Cathelicidin mRNA was also detected in rosacea skin by in situ hybridization, but not seen in normal skin. Thus, the skin of patients with rosacea, like those with other inflammatory diseases, expressed more cathelicidin than normal facial skin.

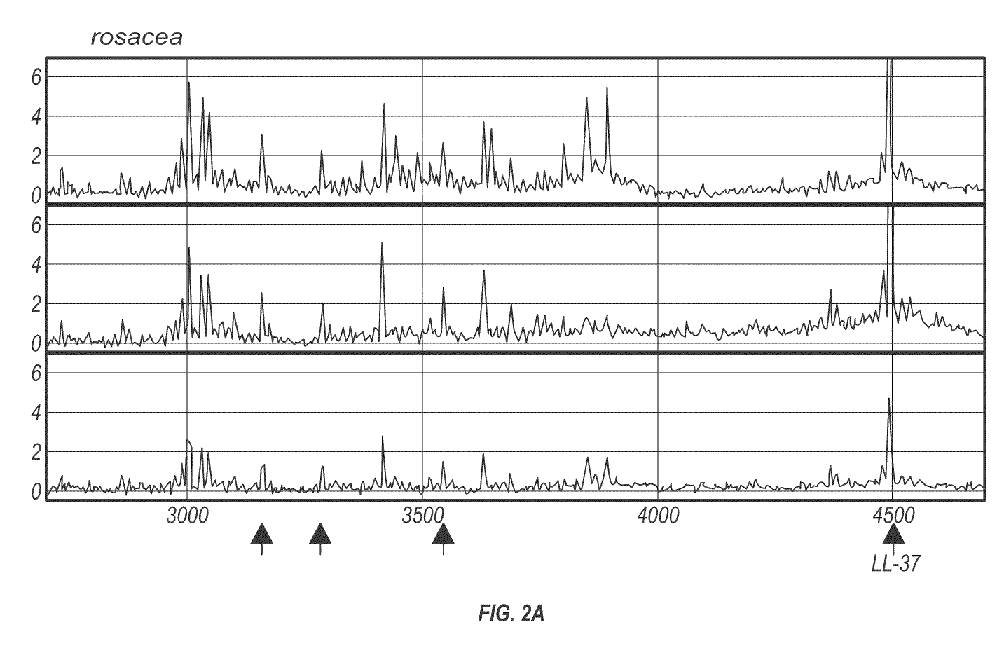

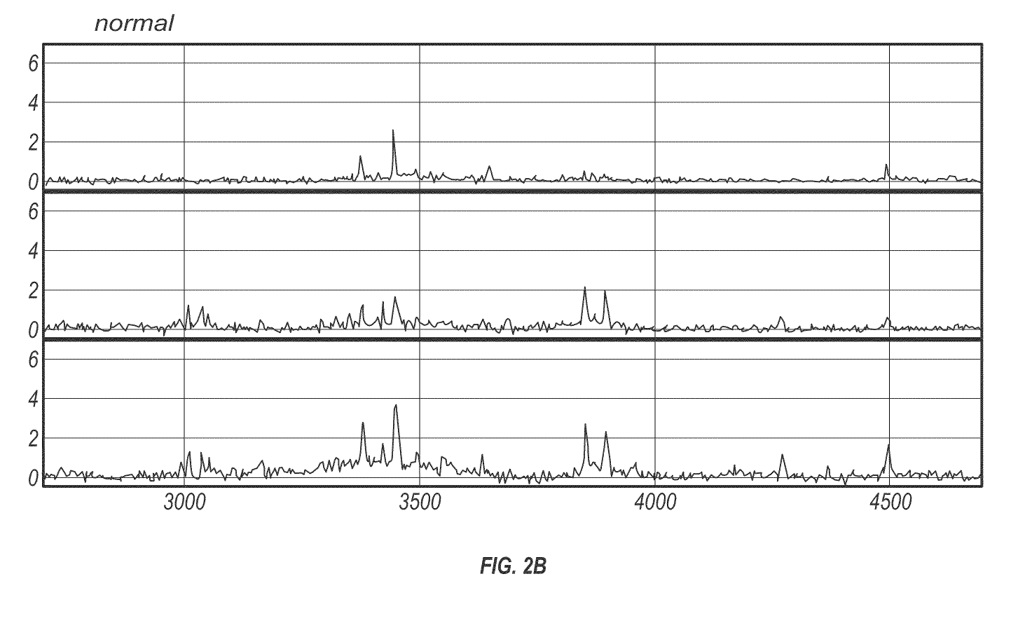

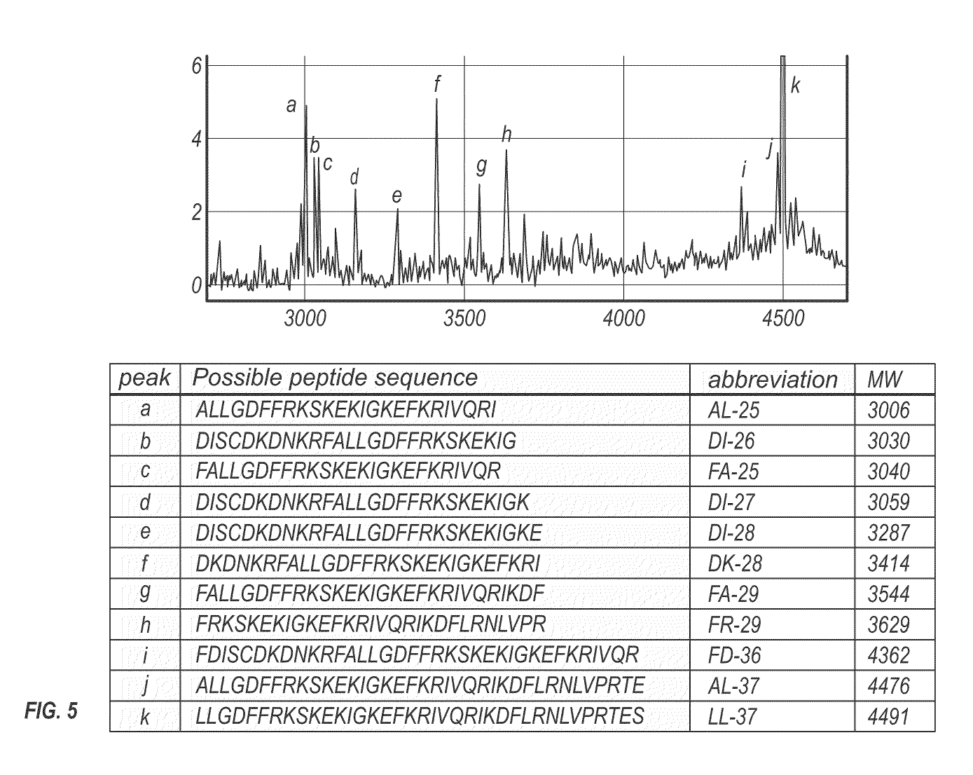

High expression of cathelicidin does not predict enhanced function since proteolytic processing of the cathelicidin precursor protein hCAP18 into active peptide is an essential step for function 24 and controls its ability to act as an antimicrobial or pro-inflammatory molecule. The mass of cathelicidin peptides from rosacea and normal skin was analyzed using SELDI-TOF-MS (Surface Enhanced Laser Desorption/Ionization, Time-of-Flight Mass Spectrometry). Cathelicidin peptide mass distributions were very similar between independent rosacea patients (FIG. 2). Samples obtained from normal facial skin were also similar to each other, but were markedly different than those in rosacea. In rosacea, LL-37 was one of the major forms of the peptide, while in normal skin this was less frequent. Furthermore, rosacea skin contained peptides of unique mass that were absent in normal skin. (arrows in FIG. 2, identified in FIG. 5). These data demonstrated that the processing of cathelicidin peptides is altered in rosacea.

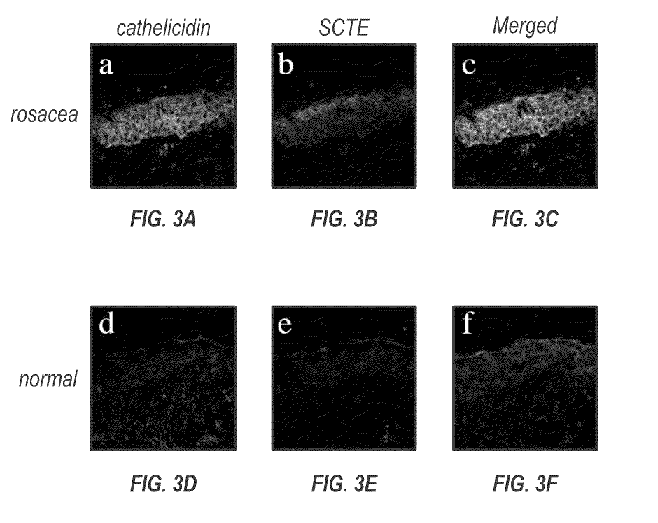

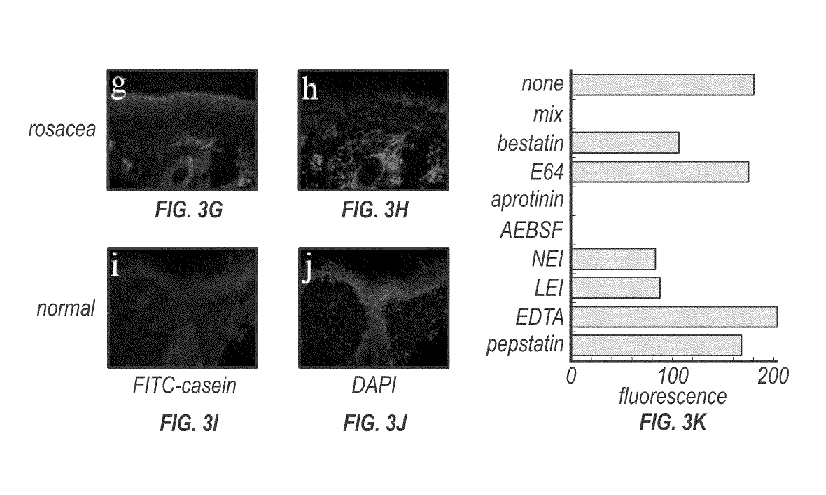

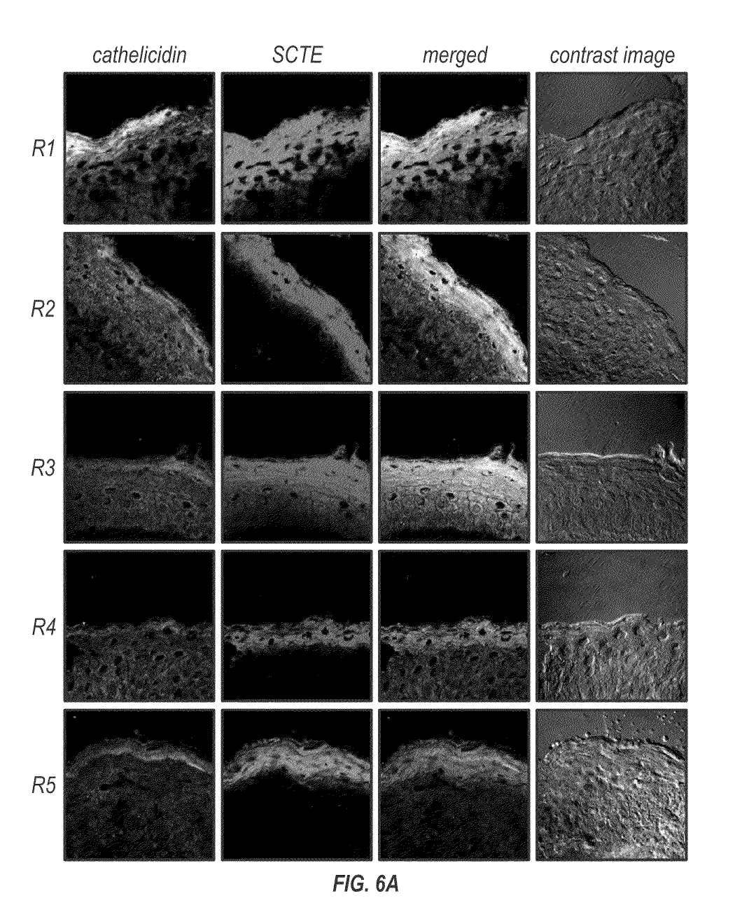

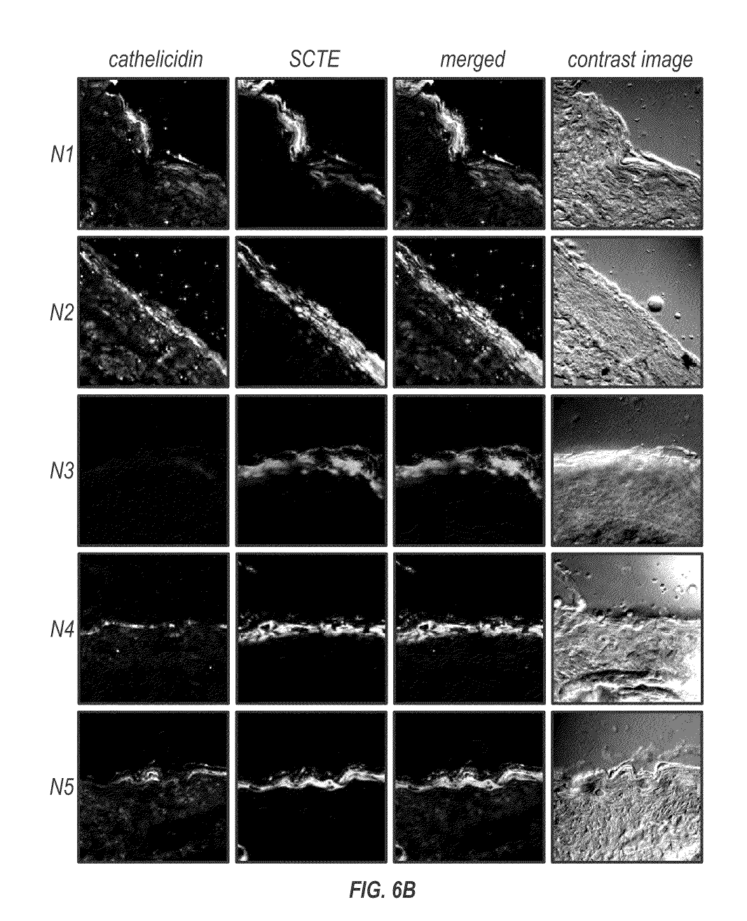

Serine proteases of the kallikrein family cleave hCAP18 to active peptides in the epidermis. Based on these results, the expression of the kallikrein SCTE (stratum corneum tryptic enzyme, KLK5) was examined to determine if it was altered in rosacea compared to normal skin. SCTE was highly expressed in rosacea and co-localized with cathelicidin in the granular and cornified layers of the epidermis (FIG. 3a-c and FIG. 6a). Some rosacea specimens also expressed SCTE in the basal layer of epidermis. By contrast, cathelicidin and SCTE were much less abundant in normal skin (FIG. 3d-f and FIG. 6b). This increase in immunoreactivity in rosacea correlated with an increase in protease activity in epidermis as determined by in situ zymography of rosacea skin compared to normal (FIG. 3g-j). Serine protease inhibitors, aprotinin and AEBSF, completely suppressed protease activity in rosacea skin (FIG. 3k). Protease activity was higher in facial skin in which rosacea symptoms appeared than at other body surface sites, and was typically undetectable in facial skin of normal patients. Taken together these data show that serine protease activity associated with increased SCTE was elevated in rosacea skin. This finding predicts the abnormal cathelicidin peptide products detected in affected individuals.

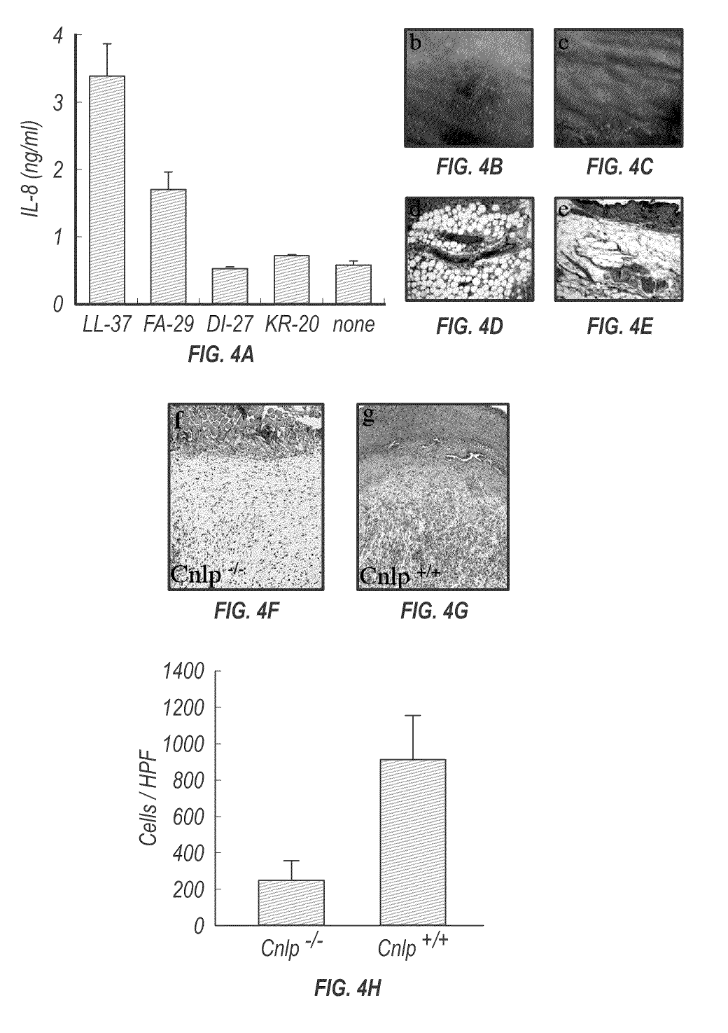

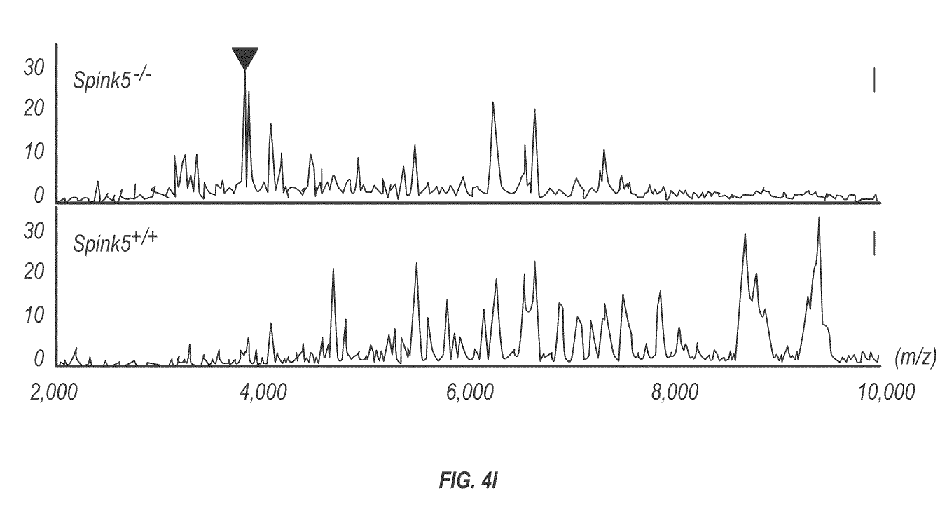

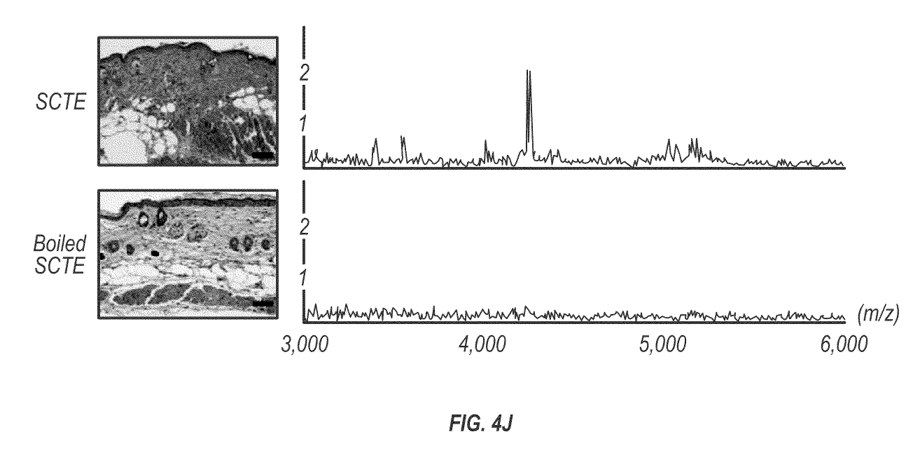

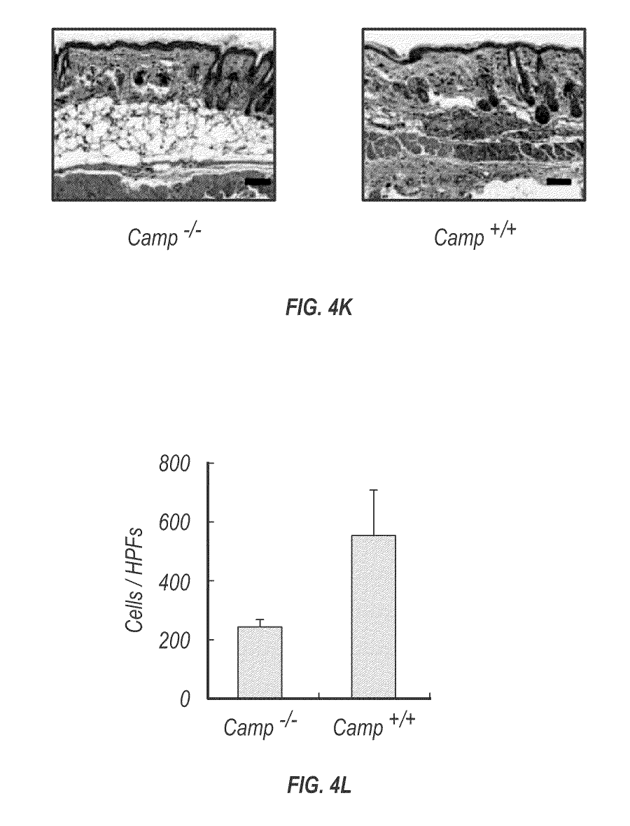

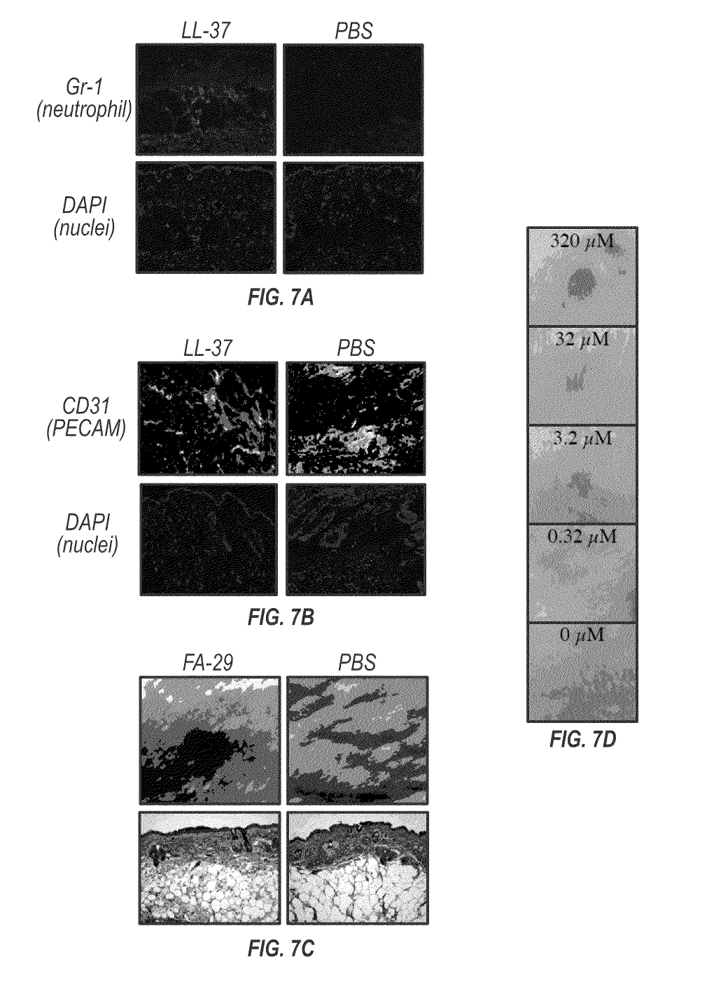

To test the consequences of the abnormal cathelicidin peptides found in rosacea, the function of these peptides was directly tested. Peptides abundant in rosacea patients, LL-37 and FA-29, induced secretion of IL-8 from cultured human keratinocytes, whereas other peptides such as DI-27 did not (FIG. 4a). KR-20, the most abundant cathelicidin detected on normal skin, did not induce cytokine release. To test function in vivo, mice were given local intradermal injections of the synthetic peptides. The concentration chosen for injection (320 μM) was selected to reflect local physiological concentrations seen in rosacea, which were measured as high as 1500 μM. LL-37 or FA-29 induced erythema and vascular dilatation after 48 hr and was characterized histologically by a neutrophilic infiltrate, thrombosis and hemorrhage (FIG. 4b-e and FIG. 7a-c). Injection of peptide KR-20 from normal skin did not induce inflammation. Inflammatory reactions to LL-37 were dose-dependent to as low as 3.2 μM (FIG. 7d) and observed equally in both Balb/C and C57/B16 mouse strains. Conversely, preventing cathelicidin release partially blocked the inflammatory response. Analysis of the inflammatory response in Cnlp −/− mice 12, showed that a deficiency of cathelicidin was accompanied by significantly less inflammation following chemical irritation than wild type mice (FIG. 4f-h). A similar decreased inflammatory infiltrate was also seen following physical abrasion of the skin.

To test how the increase in serine protease activity observed in rosacea contributes to the clinical findings of the disease, mice deficient in the gene encoding serine peptidase inhibitor Kazal-type 5 (Spink5), which do not express the serine protease inhibitor Lymphoepithelial Kazal-type-related inhibitor (LEKTI) were examined and show increased SCTE activity. Skin from Spink5^{-/-} mice had altered expression of cathelicidin peptides similar to that seen in rosacea (FIG. 4i). The main peak was GLL-34 (m/z 3,877; FIG. 4i, arrowhead), a mouse cathelicidin peptide similar in activity to human LL-37. Like LL-37, GLL-34 was not detected in wild-type mouse littermates with normal serine protease activity. These data support the hypothesis that an increase in serine protease activity leads to activation of cathelicidin. To determine whether an increase in SCTE results in greater inflammation, SCTE was injected subcutaneously in a manner similar to that used for the cathelicidin peptides themselves. The amount of SCTE in human skin is as high as 2-13 ng per mg of dry weight of skin. From the data of the protease assay, the amount of serine protease activity in lesional skin of individuals with rosacea was estimated to be as high as 500 ng of tryptic kallikrein per mg of dry weight of skin. Therefore, 1 mg of SCTE was injected twice a day for 2 d to mimic local concentrations seen in rosacea. Injection of active SCTE induced erythema and inflammatory cell infiltration accompanied by processing of cathelicidin peptide (FIG. 4g), which were not observed in control treated skin. This response to SCTE was dependent on the presence of cathelicidin, because Camp^{-/-} mouse showed considerably less cell infiltration after SCTE injection as compared with wild type mice (FIG. 4k, l). These data show that injection of SCTE increases cathelicidin processing and induces skin inflammation. Therefore, the increase in SCTE observed in rosacea would account for the pathological changes observed this disease.

Taken together the findings show cathelicidin expression can induce inflammation and vascular dilatation. Furthermore, the data show an association between the clinical findings of rosacea and the excess generation of specific cathelicidin peptides that induce changes in the skin of mice that are similar to that seen in the human disease. The production of these peptides can be explained by highly increased serine protease activity found in all patients with disease, but none of the controls.

An important concept to emerge from these observations is that both expression of the cathelicidin peptide and its processing enzymes are critical to outcome. From the current study it is unclear if the altered cathelicidin peptides are only due to the increased expression of SCTE since other serine proteases, and protease inhibitors, will also predict the final steady state accumulation of products. For example, transgenic mouse over expressing kallikrein 7 in the skin show inflammation in the dermis. Similarly, mice lacking the serine protease inhibitor LEKTI show severe epidermal disruption associated with excess active cathelicidins. In both models abnormal protease activity affects the physical barrier of the stratum corneum. In addition however, the present findings suggest that alterations of the peptide barrier by the generation of pro-inflammatory forms of cathelicidin are also an important contributor to the phenotype. Thus, skin protease activity is a critical element in regulating inflammatory signals that are generated by AMPs. This novel concept emphasizes the importance of total enzymatic activity, a process that will be modified by a variety of environmental factors such as temperature and pH. These factors all are associated with precipitating rosacea, and further support the hypothesis that these events are part of the pathophysiology of this disease.

Several lines of indirect evidence support a cause and effect relationship between the observations made here and the induction of symptoms of rosacea. First, cathelicidin peptides in the forms found in patients will induce vascular changes in animal models and in vitro, while patients without rosacea process cathelicidin into peptide forms that do not stimulate these changes but form a more effective antimicrobial shield. Secondly, as discussed earlier, cathelicidin can be induced by agents similar to those that exacerbate the disease, some of which have been associated with also increasing serine protease activity. To directly test the hypothesis that the abnormal production of cathelicidin is the cause of rosacea it would be necessary to inhibit generation of these peptides by treatment with a specific inhibitor. Unfortunately, this is not currently possible. However, clinical experience with one of the most common forms of therapy for rosacea partly accomplishes this task. Oral and topical antibiotics are effective in treating rosacea yet resistance to the antimicrobial activity of commonly used antibiotics is high, approaching 80%. This suggests the effectiveness of drugs such as tetracyclines may be due to effects not related to their antimicrobial activity. Preliminary data collected from patients treated with the minocycline has shown that this drug leads to a decrease in skin protease activity, a finding supported by previous observations of the capacity of tetracyclines to inhibit both metalloproteases and serine proteases in vitro 33-35. Thus, the effectiveness of a drug that targets cathelicidin proteolysis provides additional support for the role of these peptides in rosacea.

A consistently elevated balance between cathelicidin and its proteases can be seen in patients with rosacea, leading to accumulation of peptides that can mimic the disease. These findings suggest an entirely new direction for understanding the pathophysiology of this common disorder. Influencing the generation of AMPs through control of their expression or post-secretory processing provides direction for design of more effective therapy, and reveals a previously unsuspected role for proteolysis and AMP release in this and possibly other inflammatory disorders.

A number of embodiments of the disclosure have been described. Nevertheless, it will be understood that various modifications may be made without departing from the spirit and scope of the disclosure. Accordingly, other embodiments are within the scope of the following claims.

1. A method of treating an inflammatory disease or disorder comprising administering a cathelicidin inhibitor.

2. The method of claim 1, wherein the cathelicidin inhibitor comprises an antisense or ribozyme molecule that inhibits the expression of a cathelicidin polypeptide.

3. The method of claim 1, wherein the cathelicidin inhibitor comprises a vitamin D3 antagonist.

4. The method of claim 1, further comprising administering a serine protease inhibitor.

5. The method of claim 1, wherein the inflammatory disease or disorder is a disease or disorder of the skin.

6. The method of claim 5, wherein the disease or disorder of the skin is acne or rosacea.

7. A method of treating rosacea or acne comprising administering a serine protease inhibitor.

8. The method of any one of claims 1 or 7, wherein the administering is by topical application.

9. A composition comprising a cathelicidin inhibitor or a cathelicidin and protease inhibitor.

10. The composition of claim 9, wherein the cathelicidin inhibitor comprises an antisense or ribozyme molecule that inhibits the expression of a cathelicidin polypeptide.

11. The composition of claim 9, wherein the cathelicidin inhibitor comprises a vitamin D3 antagonist.

12. The composition of claim 9, wherein the composition further comprises a pharmaceutically acceptable carrier.

13. The composition of claim 9, formulated for topical administration.

14. A method for determining whether a subject has or is at risk of having rosacea or acne comprising determining the level of a cathelicidin polypeptide and/or serine protease in a sample from the subject, wherein an elevate level of cathelicidin and/or a serine protease is indicative of rosacea or risk of having rosacea.

15. The method of claim 14, wherein the cathelicidin polypeptide is LL-37 and/or FA-29.

16. The method of claim 14, wherein the serine protease is kallikrein SCTE.

17. The method of claim 14, wherein the method comprises measuring the level of both cathelicidin and a serine protease.

18. The method of claim 14, wherein the cathelicidin comprises the LL-37 domain of cathelicidin.

19. A method for determining whether a subject has or is at risk of having rosacea comprising determining a polypeptide mass in a sample from the subject, wherein an elevate level of mass is indicative of rosacea or risk of having rosacea.

20. A kit comprising a reagent useful for identifying the level of cathelicidin and/or serine protease in a skin sample.

21. The kit of claim 20, wherein the kit is compartmentalized to receive one or more of (i) an oligonucleotide for detection of a cathelicidin or fragment thereof and a serine protease; or (ii) an antibody for detection of cathelicidin or a fragment thereof and a serine protease.


SEQUENCE LISTING (15 sequences)

SEQ ID NO: 1 - DNA, 739 bp, homo sapiens
  CDS (141)..(653)
taaagcaaaccccagcccacaccctggcaggcagccagggatgggtggatcaggaaggctcctggttgggcttttgcatcaggctcaggctgggcataaaggaggctcctgtgggctagagggaggcagacatggggaccatgaagacccaaagggatggccactccctggggcggtggtcactggtgctcctgctgctgggcctggtgatgcctctggccatcattgcccaggtcctcagctacaaggaagctgtgcttcgtgctatagatggcatcaaccagcggtcctcggatgctaacctctaccgcctcctggacctggaccccaggcccacgatggatggggacccagacacgccaaagcctgtgagcttcacagtgaaggagacagtgtgccccaggacgacacagcagtcaccagaggattgtgacttcaagaaggacgggctggtgaagcggtgtatggggacagtgaccctcaaccaggccaggggctcctttgacatcagttgtgataaggataacaagagatttgccctgctgggtgatttcttccggaaatctaaagagaagattggcaaagagtttaaaagaattgtccagagaatcaaggattttttgcggaatcttgtacccaggacagagtcctagtgtgtgccctaccctggctcaggcttctgggctctgagaaataaactatgagagcaatttcaaaaaaaaaaaaaaaaaaaaaaaaa

SEQ ID NO: 2 - protein, 170 aa, homo sapiens
MKTQRDGHSLGRWSLVLLLLGLVMPLAIIAQVLSYKEAVLRAIDGINQRSSDANLYRLLDLDPRPTMDGDPDTPKPVSFTVKETVCPRTTQQSPEDCDFKKDGLVKRCMGTVTLNQARGSFDISCDKDNKRFALLGDFFRKSKEKIGKEFKRIVQRIKDFLRNLVPRTES

SEQ ID NO: 3 - protein, 37 aa, Artificial Sequence
  Artificial polypeptide sequence derived from Mus musculus
FKKISRLAGLLRKGGEKIGEKLKKIGQKIKNFFQKLV

SEQ ID NO: 4 - protein, 25 aa, Artificial Sequence
  Partial Peptide Sequence from TOF-MS
ALLGDFFRKSKEKIGKEFKRIVQRI

SEQ ID NO: 5 - protein, 27 aa, Artificial Sequence
  Partial Peptide Sequence from TOF-MS
DISCDKDNKRFALLGDFFRKKSKEKIG

SEQ ID NO: 6 - protein, 25 aa, Artificial Sequence
  Partial Peptide Sequence from TOF-MS
FALLGDFFRKSKEKIGKEFKRIVQR

SEQ ID NO: 7 - protein, 27 aa, Artificial Sequence
  Partial Peptide Sequence from TOF-MS
DISCDKDNKRFALLGDFFRKSKEKIGK

SEQ ID NO: 8 - protein, 28 aa, Artificial Sequence
  Partial Peptide Sequence from TOF-MS
DISCDKDNKRFALLGDFFRKSKEKIGKE

SEQ ID NO: 9 - protein, 28 aa, Artificial Sequence
  Partial Peptide Sequence from TOF-MS
DKDNKRFALLGDFFRKSKEKIGKEFKRI

SEQ ID NO: 10 - protein, 29 aa, Artificial Sequence
  Partial Peptide Sequence from TOF-MS
FALLGDFFRKSKEKIGKEFKRIVQRIKDF

SEQ ID NO: 11 - protein, 29 aa, Artificial Sequence
  Partial Peptide Sequence from TOF-MS
FRKSKEKIGKEFKRIVQRIKDFLRNLVPR

SEQ ID NO: 12 - protein, 36 aa, Artificial Sequence
  Partial Peptide Sequence from TOF-MS
FDISCDKDNKRFALLGDFFRKSKEKIGKEFKRIVQR

SEQ ID NO: 13 - protein, 37 aa, Artificial Sequence
  Partial Peptide Sequence from TOF-MS
ALLGDFFRKSKEKIGKEFKRIVQRIKDFLRNLVPRTE

SEQ ID NO: 14 - protein, 37 aa, Artificial Sequence
  Partial Peptide Sequence from TOF-MS
LLGDFFRKSKEKIGKEFKRIVQRIKDFLRNLVPRTES

SEQ ID NO: 15 - protein, 20 aa, Artificial Sequence
  Artificially synthesized peptide
KRIVQRIKDFLRNLVPRTES